(19) United States
(12) Patent Application Publication
(10) Pub. No.: US 2026/0108551 A1
(43) Pub. Date: Apr. 23, 2026

(54) IRON COMPLEX COMPOUNDS FOR SUBCUTANEOUS USE IN THERAPY OF IRON DEFICIENCY IN COMPANION ANIMALS

(71) Applicant: PHARMACOSMOS HOLDING A/S, Holbaek (DK)

(72) Inventors: Hans B. ANDREASEN, Holbaek (DK); Tobias S. CHRISTENSEN, Roskilde (DK); Simon M. GULDBERG, Hornbaek (DK)

(21) Appl. No.: 18/294,572

(22) PCT Filed: Aug. 3, 2022
(86) PCT No.: PCT/EP2022/071875

§ 371 Date: Feb. 2, 2024

(30) Foreign Application Priority Data
Aug. 3, 2021 (EP) 21189392.0

(51) Int. Cl. A61K 31/715; A61K 9/00; A61P 7/06

(52) U.S. Cl. CPC A61K 31/715; A61K 9/0021; A61P 7/06

(57) ABSTRACT

The present invention relates to iron complex compounds for subcutaneous use in a method of therapy, e.g., for iron deficiency, particularly of iron deficiency anaemia, in a companion animal and to pharmaceutical compositions for subcutaneous administration, comprising an iron complex compound and a pharmaceutically acceptable carrier. The present invention relates more particularly, although not exclusively, to iron octasaccharide complexes, pharmaceutical compositions comprising an iron octasaccharide complex, and the iron octasaccharide complexes for use in a method of therapy of iron deficiency, particularly of iron deficiency anaemia, in a human or non-human subject.

### FIELD OF THE INVENTION

The present invention relates to iron complex compounds for subcutaneous use in a method of therapy of iron deficiency in a companion animal and to pharmaceutical compositions for subcutaneous administration, comprising an iron complex compound and a pharmaceutically acceptable carrier. The present invention relates more particularly, although not exclusively, to iron octasaccharide complexes, pharmaceutical compositions comprising an iron octasaccharide complex, and the iron octasaccharide complexes for use in a method of therapy of iron deficiency in a human or non-human subject.

### BACKGROUND

Anaemia is a relatively common clinical sign and laboratory abnormality seen in dogs and also other companion animals. Of all dogs presented in a clinical setting to U.S. Banfield hospitals in 2005, between 3% and 11% were anaemic (Lund, 2007). Of these anaemic dogs, a certain share is believed to be anaemic due to deficiency in iron (iron deficiency; ID), resulting in an estimated prevalence of canine iron deficiency anaemia (IDA) of 20-110 dogs in every 10,000 dogs. With a U.S. dog population of about 90 million (American Pet Products Association, National Pet Owners Survey 2016), this translates into 180,000 to 990,000 dogs with IDA. Thus, IDA in dogs is similarly prevalent and important as in humans.

The field of clinical haematology is key to most disorders in companion animals and comprises anaemias caused by blood loss, haemolysis, bone marrow disorders, bleeding disorders, hematopoietic toxicities, infections, and cancer. It addresses routine and specific diagnostics of red blood cells (RBC), white blood cells, platelets, and coagulation factors, and the supportive and specific therapies of anaemia including transfusion therapy.

During erythropoiesis, RBCs are produced from pluripotent stem cells in bone marrow which undergo proliferation and maturation over a 7-10 day period before a mature RBC is released into the circulation. Erythropoiesis is mostly regulated by the renal hormone erythropoietin which is synthesised and released from kidney cells when there is renal hypoxia. Haemoglobin is synthesised during the later stages of erythroid maturation from burst-forming units and (meta-) rubricytes to reticulocytes. In order for haemoglobinization to occur, iron needs to be present in the marrow.

In a healthy dog, for instance, red blood cells make up 36-58% of the blood volume. They have no nucleus and mitochondria and have therefore a finite lifespan of approximately 100 days, and are destroyed extravascularly by macrophages. The primary function of RBCs is to serve as oxygen carrier. Each RBC contains about 33% haemoglobin (Hb) which is composed of 4 globin chains and one central heme containing iron. Iron in the ferrous (Fe2+) form can bind oxygen and depending on the oxygen tension will bind (lungs) oxygen to haemoglobin or release (peripheral tissue) oxygen from Hb. Tissue oxygenation is essential for energy production and all cell functions.

The term anaemia stems from the Greek words “an” (without) and “heima” (blood). Anaemia is a clinical sign in animals or a clinical symptom in humans. A low Hb concentration and/or low haematocrit (Hct)/packed cell volume (PCV) indicate the presence of anaemia. While in human medicine, Hb concentrations are preferred, in veterinary medicine Hct/PCV have been most commonly used (albeit those parameters are less accurate). Mechanistically anaemias are divided into haemolytic anaemias, blood loss anaemias, and reduced or inefficient erythropoiesis. With haemolysis, Hb breakdown products of lysed RBCs are recycled and plenty of iron is generally available. Blood loss may be internal or external, may be local due to a laceration or multifocal due to haemostatic disorders; and with chronic external blood loss iron IDA will ensue. Also, reduced or inefficient erythropoiesis can have many causes such as toxicities, infections, cancer, kidney disease and nutritional deficiencies such as iron and vitamin B12 deficiency (Giger, 2005; Weiss, 2010).

Dogs, for instance, have approximately 10-50 mg iron/kg bodyweight, and most is located in erythrocytes as Hb; in fact, 1 mg iron is in every 2 mL of blood. In addition, myoglobin in muscle tissues and many important enzymes in all cells, such as cytochromes involved in energy and drug metabolism, represent heme proteins and contain iron. Iron is transported via transferrin in blood and stored as a soluble mobile fraction (ferritin) and insoluble fraction (hemosiderin), primarily in the spleen, liver, and bone marrow, depending on the amount of iron present (Olver et al., 2010; Cohen-Solal et al., 2014). Transferrin represents the iron transporter in plasma and is normally 20-60% saturated.

Under physiological conditions, iron losses through the gut, urine, and skin are negligible, amounting to less than 1-2 mg/day in dogs. Iron is acquired through diets with the meat containing canine diets being rich in iron. Because too much iron can be toxic, iron balance is carefully regulated by intestinal absorption. The mechanisms of iron absorption in the duodenum have recently been elucidated with the hepatic hormone hepcidin being the main inhibitory regulator. Iron absorption increases with decreased iron storage and increased erythropoietic activity and is associated with low hepcidin concentrations. In the presence of high serum iron concentrations, hepcidin is released from the liver and forms a complex with ferroportin to reduce intestinal iron absorption (Olver et al., 2010).

While companion animals with IDA such as dogs are commonly managed with blood transfusions and parenteral and oral iron supplements in addition to the correction of the cause of IDA, there are no detailed reports on efficacy and safety of treatments in canine medicine. The proper development and regulatory approval of safe and effective parenteral iron drugs would be of great benefit and value to dogs and other companion animals with IDA.

As far as oral iron drugs and iron food supplements are concerned, it can be summarised—albeit with little solid evidence—that ferrous sulphate, gluconate or fumarate drugs at a dose of 5 mg/lb per day or 100 to 300 mg per day per dog may be effective provided that a) the gastrointestinal iron uptake is intact, b) gastrointestinal side-effects are absent or tolerable (such as vomiting and diarrhoea), and c) the daily dosage is adhered to for several weeks to months.

As for parenteral iron drugs, there are presently no such drugs approved in the U.S. and many other countries for the prevention, treatment, management or control of IDA in dogs. Despite this situation, use of a human or baby pig-approved iron dextran product in dogs has been described in several sources.

The variety of recommendations reflect this situation:
Plumb's Veterinary Drug Handbook (Plumb, 2008): “For iron deficiency anemia, iron dextran 10-20 mg/kg once, followed by oral therapy with ferrous sulfate.”
Textbook of Veterinary Internal Medicine (Giger, 2005): “To replenish iron stores in context of iron deficiency anemia secondary to disease, if oral iron replacement is deemed inappropriate or insufficient or a gastrointestinal disorder prevents iron absorption, parenteral iron may be administered. Up to 2 mL iron dextran complex (50 mg/mL) intramuscularly daily.” I.e. up to 100 mg iron daily or up to 20 mg/kg in a 5 kg/11 lb dog, and lower specific dose (mg/kg/day) in larger dogs.
Small Animal Internal Medicine (Nelson & Couto, 1998): For iron deficiency anemia, “Intramuscularly administered iron dextran can also be given at a dosage of 10 mg/kg once to twice a week. This form of iron therapy is associated with pain at the site of injection and the potential for anaphylactic reactions.”
Blackwell's Five-Minute Veterinary Consult (Weiser, 2015): For iron deficiency anemia, “Initiate iron therapy with injectable iron. Iron dextran—a slowly released form of injectable iron; one injection (10-20 mg/kg IM) followed by oral supplementation.”

Belfer® (an iron dextran complex 100 mg/mL, bela-pharm GmbH & Co. KG) is approved for use in a variety of animal species, however, the safety and efficacy of Belfer® has not been studied in all species for which it is approved—in particular, it appears that it has previously not been studied in cats and dogs. Nonetheless, it is approved for use in dogs in several countries. The approved dosage and administration for dogs is 1-2 mg iron per kg bodyweight (i.e. 0.01-0.02 mL per kg bodyweight) given as an intramuscular injection. This is a rather low and likely a subtherapeutic dose for treating IDA in dogs.

A safe parenteral iron drug for dogs with IDA administered at the right dose and in the right manner would have the key advantage that the therapeutic effect of replenishing iron stores and raising the Hb concentration—and thus also Hct/PCV—occurs very rapidly.

A few published case reports exist on intramuscular iron dextran injection in doses of 10 to 20 mg/kg in dogs for treatment or management of IDA. Examples of such case reports are:
Single iron dextran injection 13 mg/kg in a Golden retriever (Cook & Kvitko-White, 2014).
Single iron dextran injection 15 mg/kg in a mixed breed dog (Thrall & Gillespie, 2011).
3 iron dextran injections of 10 mg/kg within one week in two dogs (Harvey et al., 1982).
1-2 iron dextran injections of 20 mg/kg in 7 dogs, 6-13 days apart (Fry & Kirk, 2006).

Another report describes infusion of an iron oligosaccharide in healthy dogs:
Infusion of iron-(11l)-hydroxide oligosaccharide at doses of 7.1 and 21.3 mg/kg (Preusser et al, 2005).

However, there are presently no parenteral iron drugs that have been specifically developed for the therapy of iron deficiency in companion animals.

The present invention has been devised in light of the above considerations.

### SUMMARY OF THE INVENTION

In one aspect, the present invention relates to an iron complex compound for subcutaneous use in a method of therapy of iron deficiency in a companion animal.

In a first embodiment of said first aspect, the companion animal is a dog.

In a second embodiment of said first aspect, the method comprises administering a dose of 20 mg elemental iron per kg bodyweight.

In a second aspect, the present invention relates to a pharmaceutical composition for subcutaneous administration, comprising an iron complex compound and a pharmaceutically acceptable carrier.

In a first embodiment of said second aspect, the pharmaceutical composition is a ready-to-use injectable composition.

In a second embodiment of said second aspect, the pharmaceutical composition comprises 100 mg/mL of elemental iron.

In a further embodiment of said first and second aspect, the iron complex compound is an iron oligoisomaltose complex or an iron oligoisomaltoside complex. In a particular embodiment, the iron complex compound is an iron oligosaccharide complex comprising iron complexed with an oligoisomaltoside, wherein (i) the oligoisomaltoside has a weight average molecular weight in the range of 850 to 1,150 Da; (ii) the content of monosaccharide and disaccharide is less than 10.0% by weight of the oligoisomaltoside; (iii) the fraction with more than 9 monosaccharide units is less than 30% by weight of the oligoisomaltoside; (iv) at least 40% by weight of the molecules have 3-6 monosaccharide units; (v) the “apparent” peak molecular weight (Mp) of the complex is in the range of 130,000 to 180,000 Da; (vi) the dispersity (Mw/Mn) of the complex is in the range of 1.05 to 1.4; and (vii) the amount of reducing sugar is 2.5% or less by weight of the oligoisomaltoside. Alternatively and preferably when considered in the context of subcutaneous administration, the iron complex compound is an iron octasaccharide complex comprising iron complexed with an octasaccharide, wherein (i) the octasaccharide has a weight average molecular weight in the range of 1,150 to 1,350 Da; (ii) the content of monosaccharide and disaccharide is less than 10.0% by weight of the octasaccharide; (iii) the fraction with more than 9 monosaccharide units is less than 40% by weight of the octasaccharide; (iv) at least 40% by weight of the molecules have 6-10 monosaccharide units; (v) the “apparent” peak molecular weight (Mp) of the octasaccharide complex is in the range of 125,000 to 185,000 Da; (vi) the dispersity (Mw/Mn) of the complex is in the range of 1.05 to 1.4; and (vii) the amount of reducing sugar is 2.5% or less by weight of the octasaccharide.

In a third aspect, the present invention relates to said iron octasaccharide, pharmaceutical composition comprising said iron octasaccharide and a pharmaceutically acceptable carrier, and the iron octasaccharide complex for use in a therapeutic method, more specifically for use in a method of therapy of iron deficiency such as iron deficiency anaemia in a human or non-human subject.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows baseline-corrected serum iron concentrations (μmol/L; mean+/−SD) after administration of an intra-muscular (IM) dose (20 mg/kg=T4) or a subcutaneous (SC) dose (20 mg/kg=T1; 60 mg/kg=T2; and 100 mg/kg=T3) of iron oligoisomaltoside complex.

FIG. 2 shows change from baseline ferritin (ng/mL; mean+/−SD) after administration of an IM dose (20 mg/kg=T4) or an SC dose (20 mg/kg=T1; 60 mg/kg=T2; and 100 mg/kg=T3) of iron oligoisomaltoside complex.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### Definitions

In order that the present description may be more readily understood, certain terms are first defined. Additional definitions are set forth throughout the detailed description.

“Therapy” refers to any type of intervention or process performed on, or the administration of an active agent to, the subject with the objective of reversing, alleviating, ameliorating, inhibiting, slowing down, or preventing the onset of, the progression, development, severity, or recurrence of a clinical sign or symptom, complication, condition, or biochemical indicia associated with a disease or disorder. “Therapy” as used herein particularly includes the prevention, control, treatment, or management of a disease or disorder such as iron deficiency or iron deficiency anaemia. According to the invention, the treatment or management of ID and especially of IDA represents a particular embodiment.

A “subject” includes any human or non-human animal. The term “non-human animal” includes, but is not limited to, vertebrates such as non-human primates, companion animals, in particular canines, felines, equines, and camels, and rodents such as mice, rats and guinea pigs. The term “non-human animal” also includes livestock, such as swine, goat, sheep, and cattle. The terms, “subject” and “patient” are used interchangeably herein.

The term “companion animal” refers to an animal suitable to be a companion to humans. In some embodiments, a companion animal is a canine (such as a dog), a feline (such as a cat), an equine (such as a horse), or a camel. In some embodiments, a companion animal species is a small mammal, such as a dog, cat, rabbit, ferret, guinea pig, rodent, etc. In some embodiments, a companion animal species is a farm animal, such as a horse or a llama. In some embodiments, a companion animal species is an animal used in competition, for instance, a race animal, such as a race dog or a racehorse. Companion animals are to be distinguished from livestock, such as swine, goat, sheep, cattle, etc.

A “therapeutically effective amount” or “therapeutically effective dose” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human or animal subjects during clinical trials, in animal model systems predictive of efficacy in humans, or by assaying the activity of the agent in in vitro assays.

The terms “pharmaceutical formulation” and “pharmaceutical composition” refer to a preparation which is in such form as to permit the biological activity of the active ingredient(s) to be effective, and which contains no additional components that are unacceptably toxic to a subject to which the formulation would be administered.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

For the purpose of this text, when specifying a dose in mg or g of an iron complex compound, consistent with the practice in the literature, the value refers to the amount of elemental iron provided in mg or g.

### Therapeutic Methods

Described herein are therapeutic methods, i.e., therapy of iron deficiency which comprise administering an iron complex compound according to defined administration regimens and/or to particular subjects. Accordingly, the present invention also relates to an iron complex compound for use in said methods, the use of an iron complex compound for therapy of iron deficiency and/or the use of an iron complex compound in the manufacture of a medicament for therapy of iron deficiency.

The methods of the invention are typically performed on a subject in need thereof. A subject in need of the methods of the invention is a subject having, diagnosed with, suspected of having, or at risk for developing iron deficiency (ID). In some embodiments, the iron deficiency is iron-deficiency anaemia. Iron-deficiency anaemia (IDA) develops when iron stores are depleted. Subjects who suffer from ID may have IDA; subjects with IDA necessarily suffer from ID. Methods to diagnose ID and IDA are well established in the art and commonly used in clinical practice.

Subjects having, diagnosed with, suspected of having, or at risk for developing ID or IDA will be given parenteral iron in the form of an iron complex compound if oral iron is not tolerated or not effective or cannot be used in the subject, e.g., subjects who (i) have intolerance to oral iron or (ii) have had unsatisfactory response to oral iron or (iii) where the use of oral iron is not practically completed for a required duration of time (i.e., in a case of non-adherence/non-compliance to the oral iron treatment course). Another situation where intravenous, subcutaneous or intramuscular iron is indicated is a need to deliver iron rapidly, i.e., when there is a clinical need for rapid repletion of iron stores.

### Subjects to be Administered Therapy—Companion Animals

The invention relates in particular to the therapy of iron deficiency in companion animals, such as a canine (such as a dog), a feline (such as a cat), or an equine (such as a horse), especially canines and felines, such as dogs and cats. Preferably, the companion animal according to the invention is a dog.

The invention also relates to the therapy of iron deficiency in a human or non-human subject.

### Iron Deficiency and Anaemia

Iron deficiency anaemia states ensue when iron is unavailable during erythropoiesis either because of inadequate or depleted body iron stores—absolute IDA—or inability to mobilize iron from otherwise adequate body iron stores—functional IDA.

Functional iron deficiency anaemia is observed with infections, inflammation and cancer (Naigamwalla et al., 2012). Here, hepcidin causes a sequestration of iron in the insoluble form hemosiderin in macrophages of the liver, spleen and bone marrow. Hence iron cannot be mobilised for erythropoiesis (Dignass, 2015), and a functional IDA ensues.

In absolute IDA, body iron stores have become depleted over a period of weeks as either a consequence of chronic blood loss or low dietary iron uptake. Chronic external blood loss is the major cause of iron deficiency anaemia in dogs. Severe parasitism due to ectoparasitism, such as flea and less common tick and maggot infestation, and endoparasitism, such as hookworms and less commonly whipworms, can cause substantial blood loss. Chronic and intermittent gastrointestinal (GI) blood loss is a frequent cause of external chronic or recurrent external blood and thereby iron loss may result from bleeding GI neoplasms, such as leiomyomas/-sarcomas and lymphoma, but also ulcerogenic drugs (glucocorticosteroids, non-steroidal anti-inflammatory agents, chemotherapeutics). In addition, chronic inflammatory bowel disease (IBD) may be associated with substantial blood loss. As each regular blood collection of ˜450 ml blood from a >23 kg blood donor subject removes ˜200 mg iron from the body, overzealous blood donations and frequent phlebotomies for diagnostic purposes in small-sized subjects may also lead to iron deficiency states.

While the above triggers may result in major bleeding despite normal haemostasis, haemostatic disorders may cause further chronic and/or recurrent severe bleeding with ensuing IDA. These include hereditary coagulopathies, thrombocytopenias, thrombopathias and von Willebrand disease.

In neonatal to juvenile subjects, e.g., puppies and young dogs, the iron needed for rapid growth and erythropoiesis may exceed the supply available from diet and body stores. In fact, iron stores in neonates are generally low, and mother's milk is low in iron. Nonetheless, IDA is typically only seen in puppies together with chronic external blood loss from endo- and ectoparasitism.

Conditions causing iron deficiency anaemia (IDA) thus include, but are not limited to, bleeding into gastrointestinal (GI) tract, which may be due to endoparasites (e.g. hook- and whipworms), gastrointestinal neoplasm (e.g. leiomyoma/-sarcoma), gastric ulceration (e.g. induced by drugs-, such as glucocorticoids, non-steroidal anti-inflammatory agents, or chemotherapeutics), inflammatory bowel disease (IBD), or other severe GI infiltrates including cancers; other external haemorrhage, such as epistaxis (nasal tumour, foreign body, infection), haemorrhagic cystitis; kidney/bladder neoplasms, blood donation (in particular when frequently repeated), ectoparasites (e.g. fleas, ticks, maggots), haemorrhage from skin lesions, surgery/trauma, or uterine and vaginal blood loss; haemostatic disorders, such as coagulopathy, thrombocytopenia, thrombocytopathia, thrombopathia, von Willebrand disease, or vasculopathy; inadequate dietary iron, for instance in nursing and weaned puppies, due to iron-depleted diet (meat-free), or iron absorption defects (e.g. iron resistant iron deficiency anaemia); and other conditions, such as chronic kidney disease (CKD).

Conditions that cause IDA may be chronic or non-chronic. Conditions of chronic or ongoing external blood loss that may cause IDA can result from etiologies that are difficult to address and treat. Examples are cancer, gastrointestinal (GI) bleeding (e.g. induced by a non-resectable tumour), or an inflammatory condition, such as inflammatory bowel disease (IBD). Such conditions may be only manageable or controllable in the sense that their effects may be delayed or reduced by employing therapies that manage or control the disease or condition. Such diseases or conditions may be described as being chronic, ongoing, intermittent, and/or non-treatable. In certain embodiments, IDA caused by a chronic disease or condition is managed or controlled by administering more than one dose of an iron complex compound to the subject (e.g. by repeat dosing).

IDA may also be caused by one or more underlying diseases or conditions that are treatable and non-chronic, such as ectoparasites (e.g. fleas, ticks, maggots) and endoparasites (e.g. hookworms and whipworms), gastrointestinal (GI) bleeding (e.g. induced by resectable tumours and neoplasms, ulcers, lacerations, non-steroidal anti-inflammatory drug-induced bleeding), malabsorption of dietary iron, inadequate nutrition, or other external hemorrhage (e.g. urinary bleeding, cystitis, trauma, surgical blood loss, blood donation). Such conditions may be treated. Such diseases or conditions may be described as being acute, non-chronic, and/or treatable. In certain embodiments, IDA caused by a non-chronic disease or condition is treated by administering a single dose of an iron complex compound to the subject.

The frequency with which said conditions occur in companion animals varies with the species. In cats, for instance, CKD can be seen at any age, but is most commonly seen in middle to old-aged cats (those over 7 years), and it becomes increasingly common with age. Therefore, cats with CKD represent a particular group of subjects that are amenable to therapy according to the invention. Other particular groups of subjects that are amenable to therapy according to the invention will be apparent to those skilled in veterinary medicine.

### Clinical Signs and Symptoms of Iron Deficiency and Iron Deficiency Anaemia

The diagnosis of iron deficiency and iron deficiency anaemia is based on an evaluation of the subject's clinical history and presenting clinical signs or symptoms (physical examination) combined with haematological analyses.

Symptoms of iron deficiency in humans can occur before the condition has progressed to iron-deficiency anaemia. Symptoms of iron deficiency can include, for example, fatigue, dizziness, pallor, hair loss, Irritability, weakness, pica, brittle or grooved nails, Plummer-Vinson syndrome (painful atrophy of the mucous membrane covering the tongue, pharynx and esophagus), impaired immune function, pagophagia, and restless legs syndrome, among others.

In companion animals such as dogs, iron deficiency and iron deficiency anaemia develop over weeks to months and are often insidious, allowing remarkable adaptation in the animal. Except for the specific signs of external bleeding, including the intestinal tract, the clinical signs are rather unspecific and depend more on the rate of progression than the extent of the anaemia. Common clinical signs of ID or IDA animals include, but are not limited to, pallor, exercise intolerance, lethargy, bounding pulse, gallop rhythm, systolic flow murmur, pica, cardiomegaly, changes in nails, and reduced muscle activity. A history of blood loss, ecto-/endoparasitism, GI disorders such as GI ulcers, neoplasm, kidney disease, or another relevant possible cause of ID and IDA are helpful indicators.

Subjects undergoing therapy according to the methods disclosed herein may experience an improvement in iron deficiency (ID). Subjects undergoing therapy according to the methods disclosed herein may experience an improvement in iron-deficiency anaemia (IDA). This improvement may occur as the total amount of iron in the body, the blood haemoglobin concentration, and/or the blood's oxygen-carrying capacity, of the subject is increased through the administration of the iron complex compound disclosed herein. In some embodiments, subjects undergoing therapy according to the methods disclosed herein experience a decrease in one or more clinical signs or in one or more symptoms of ID or IDA. In some embodiments, the decrease is temporary. In a preferred embodiment, a temporary decrease in one or more clinical signs or in one or more symptoms of ID or IDA qualifies the subject for a further dose of an iron complex compound. In other embodiments, the decrease is permanent. In some embodiments, subjects undergoing therapy according to the methods disclosed herein experience the elimination of one or more clinical signs or symptoms of ID or IDA. In some embodiments, the one or more clinical signs or symptoms of ID or IDA are selected from pallor, exercise intolerance, lethargy, bounding pulse, gallop rhythm, systolic flow murmur, pica, cardiomegaly, changes in nails, and reduced muscle activity and combinations of the foregoing.

### Iron Storage Parameters

Subjects having iron deficiency may demonstrate low or inadequate markers of systemic iron status. This means that such subjects may not have sufficient iron stored within their bodies to maintain proper iron levels. Most well-nourished, healthy dogs, for instance, may have a few grams of iron stored within their bodies. Some of this iron is contained in haemoglobin, which carries oxygen through the blood. Most of the remaining iron is contained in iron binding complexes that are present in all cells, but that are more highly concentrated in bone marrow and organs such as the liver and spleen. The liver's stores of iron are the primary physiologic reserve of iron in the healthy body. Some of the body's total iron content is utilised in proteins that use iron for cellular processes such as oxygen storage (myoglobin) or performing energy-producing redox reactions (cytochrome proteins). In addition to stored iron, a small amount of iron circulates through the blood plasma bound to a protein called transferrin.

Subjects with iron deficiency first deplete the stored iron in the body. Because most of the iron utilised by the body is required for haemoglobin, iron-deficiency anaemia is the primary clinical manifestation of iron deficiency. Oxygen transport to tissues including organs is vital and severe anaemia is harmful and potentially fatal due to systemic lack of oxygen. Iron-deficient subjects will suffer, and in some instances may die, from organ damage caused by oxygen depletion well before cells run out of the iron needed for intracellular processes.

There are several markers of systemic iron status that may be measured to determine whether a subject has sufficient iron stores to maintain adequate health. These markers may be circulating iron stores, iron stored in iron-binding complexes, or both, and are also typically referred to as iron storage parameters. Iron storage parameters can include, for example, haematocrit (Hct), packed cell volume (PCV), haemoglobin concentration (Hb, also called haemoglobin level), total iron-binding capacity (TIBC), transferrin saturation (TSAT), serum iron levels, liver iron levels, spleen iron levels, and serum ferritin levels. Of these, Hct, Hb, TIBC, TSAT, and serum iron levels are commonly known as parameters measuring circulating iron stores. The liver iron levels, spleen iron levels, and serum ferritin levels are commonly referred to as parameters measuring stored iron or iron stored in iron-binding complexes.

It is noted that while the above blood parameters are determined in serum, they can likewise be determined in plasma. Serum and plasma levels correlate and can be converted into each other.

ID is typically diagnosed prior to anaemia based on early indicators of iron, such as the reticulocyte haemoglobin content or reticulocyte haemoglobin equivalent (denoted CHr and RET-He respectively, depending on the analyser used). Recent studies have investigated this parameter in canine blood and found good correlation between low CHr/RET-He and other available indicators of iron deficiency and/or iron deficiency anaemia (Fry & Kirk, 2006; Prins et al., 2009; Schaefer & Stokol, 2015; Fuchs, et al., 2017; Steinberg & Olver, C. S 2005). In some embodiments, the CHr/RET-He of an iron deficient subject is 20 pg or less. IDA is typically diagnosed based on a complete blood count measured from a blood sample from a subject. The focus is on Hb, mean corpuscular volume (MCV), mean corpuscular haemoglobin concentrations (MCHC), and erythroid parameters such as Hct/PCV and red blood cell (RBC) count; albeit there may also be a leukopenia and thrombocytosis due to IDA. The hallmark of iron deficiency anaemia, e.g., in dogs, is low haemoglobin concentration with erythrocytic microcytosis and hypochromasia (Bohn, 2013). A blood smear is used to confirm hypochromasia. The anaemia may well remain regenerative with IDA but erythropoiesis is ineffective. Thus, there is reticulocytosis as well as polychromasia with IDA.

Conveniently, automatic haematology analysers are utilised to report blood parameters including the total number of RBCs in a sample, Hb, Hct, MCV, MCHC and additional blood parameters (e.g. CHr/RET-He) by flow cytometry. In many countries, at least one of the following four parameters are measured to determine the presence of IDA: MCV, MCHC, Hb, RBC count. In some countries, CHr/RET-He may be used to determine the presence of IDA. Certain threshold values for Hb have been set, such that when a subject's haemoglobin levels fall below those values, a diagnosis of IDA may be made.

The haemoglobin concentration or level, Hb, is the total amount of haemoglobin per volume of blood. For healthy subjects, a typical Hb range is: for female humans, Hb=12.0 to 15.5 g/dL; for male humans, Hb=13.5 to 17.5 g/dL; for dogs, Hb=11.9 to 18.9 g/dL for cats, Hb=9.8 to 15.4 g/dL; and for horses, Hb=10.1 to 16.1 g/dL. In an iron-deficient subject, however, the haemoglobin concentration can be greatly reduced. In some embodiments, the Hb of an iron-deficient dog is below 6 g/dL (indicating that the dog suffers from severe IDA); in the range of 6 to 9 g/dL (indicating that the dog suffers from moderate IDA), or in the range of 9 to 11 g/dL (indicating that the dog suffers from mild IDA).

The mean corpuscular haemoglobin concentration, MCHC, is a measure of the average concentration of haemoglobin in the red blood cells, and is determined by the amount of haemoglobin protein in a given volume of packed red blood cells. It is typically calculated by dividing the haemoglobin concentration by the haematocrit. For healthy subjects, a typical MCHC range is: for female humans, MCHC=31 to 35 g/dL; for male humans, MCHC=31 to 35 g/dL; for dogs, MCHC=32.0 to 36.3 g/dL, for cats, MCHC=30 to 36 g/dL; and for horses, MCHC=35.3 to 39.3 g/dL. In an iron-deficient subject, however, the MCHC can be greatly reduced. In some embodiments, the MCHC of an iron-deficient dog is below 30 g/dL.

The mean corpuscular volume, MCV, is a measure of the average volume of a red blood corpuscle (or red blood cell). It is typically calculated by multiplying a volume of blood by the proportion of blood that is cellular (the haematocrit), and dividing that product by the number of erythrocytes (red blood cells) in that volume. For healthy subjects, a typical MCV range is: for female humans, MCV=80 to 100 g/dL; for male humans, MCV=80 to 100 fL; for dogs, MCV=66 to 77 fL, for cats, MCV=39 to 55 fL; and for horses, MCV=37.3 to 49.0 fL. In an iron-deficient subject, however, the MCV can be greatly reduced. In some embodiments, the MCV of an iron-deficient dog is below 60 fL (indicating that the dog suffers from severe IDA).

In some embodiments, subjects undergoing therapy according to the methods disclosed herein experience an increase in haemoglobin concentration. In some embodiments, the present disclosure provides methods of increasing haemoglobin concentration in a subject in need thereof, the methods comprising administering an iron complex compound to the subjects, wherein the iron complex compound provides an increase in haemoglobin concentration in the subjects.

In some embodiments, the iron complex compound provides a mean increase in haemoglobin concentration greater than 0.5 g/dL, greater than 0.6 g/dL, greater than 0.7 g/dL, greater than 0.8 g/dL, greater than 0.9 g/dL, greater than 01.0 g/dL, greater than 1.1 g/dL, greater than 1.2 g/dL, greater than 1.3 g/dL, greater than 1.4 g/dL, greater than 1.5 g/dL, greater than 1.6 g/dL, greater than 1.7 g/dL, greater than 1.8 g/dL, or greater than 1.9 g/dL at 3 weeks (21 days) after administration.

In some embodiments, the iron complex compound provides a mean increase in haemoglobin concentration of less than 7.0 g/dL, less than 6.0 g/dL, less than 5.0 g/dL, less than 4.5 g/dL, less than 4.0 g/dL, or less than 3.5 g/dL at 3 weeks (21 days) after administration.

In some embodiments, the iron complex compound provides a mean increase in haemoglobin concentration of 0.5 to 7.0 g/dL, 1.0 to 6.0 g/dL, 1.3 to 5.0 g/dL, 1.5 to 4.5 g/dL, 1.7 to 4.0 g/dL, or 1.9 to 3.5 g/dL at 3 weeks (21 days) after administration.

Mean increases in haemoglobin concentration at 1 week are expected to 0.5 to 2.0 g/dL lover than the mean increases at 3 weeks. Mean increases in haemoglobin concentration at 4 or 8 weeks are expected to be about the same as the mean increases at 3 weeks after administration.

The mean increases in haemoglobin concentration described above apply particularly to the therapy of companion animals, preferably canines and felines, most preferably dogs.

In veterinary medicine, the Hct or PCV is traditionally used as the parameter to assess anaemia and its severity (Tvedten, 2010), instead of the Hb concentration which is predominantly used in human medicine. Although Hct/PCV generally have acceptable reliability in this respect, they are less accurate than Hb. While the PCV, also known as microhematocrit, is directly measured after centrifugation of a capillary tube filled with anticoagulated blood, the Hct obtained by haematology analysers is calculated: Hct=(MCV×RBC count)+10. Nevertheless, under most conditions the Hb and PCV/Hct will be in a close relationship such that Hb (g/dL)≈⅓×Hct (%). To eliminate any potential inaccuracies, the blood Hb concentration should be considered as the primary parameter in the assessment of anaemia in companion animals.

The Hct/PCV, also referred to as packed cell volume or erythrocyte volume fraction, is the volume percentage of red blood cells in the blood. For healthy dogs, for instance, the Hct/PCV is typically 35-57% of blood volume. For healthy cats, the Hct/PCV is typically 30-45% of blood volume and for healthy horses, it is typically 27-43% of blood volume. In iron-deficient subjects, however, the Hct/PCV is often significantly depleted due to poor iron absorption and/or poor iron storage capacity. In some embodiments, the Hct/PCV of an iron-deficient dog is below 18% (indicating that the dog suffers from severe IDA); in the range of 18% to 27% (indicating that the dog suffers from moderate IDA), or in the range of 27% to 35% (indicating that the dog suffers from mild IDA).

The iron complex compound disclosed herein may be administered to subjects to increase Hct/PCV. The exact timing of administration will necessarily vary from subject to subject, depending upon, for example, the severity of the iron deficiency experienced by the subject, the level of iron absorption the subject is or is not experiencing, and the judgment of the treating health care professional. In some embodiments, the present disclosure provides methods of increasing Hct/PCV in a subject in need thereof, the methods comprising administering an iron complex compound to the subject, wherein the iron complex compound provides for an increase in the Hct/PCV of the subject. In some embodiments, the increase is from 1% to 30%, from 1% to 15%, from 1% to 12%, from 1% to 10%, from 1% to 9%, from 1% to 8%, from 1% to 7%, from 1% to 6%, from 1% to 5%, from 1% to 4%, from 1% to 3%, or from 1% to 2%.

In addition to these parameters, measurement of serum ferritin can aid in the diagnosis of IDA. The liver's stores of ferritin are the primary source of stored iron in the body. Ferritin is an intracellular protein that stores iron and releases it in a controlled fashion. Medically, the amount of ferritin present in a blood sample and/or in a sample of liver tissue reflects the amount of iron that is stored in the liver (although ferritin is ubiquitous and can be found in many other tissues within the body in addition to the liver). Ferritin serves to store iron in the liver in a nontoxic form and to transport it to areas where it is required. A low ferritin level is generally indicative of iron deficiency anaemia. However, a normal ferritin level does not rule out IDA because ferritin is also an acute phase protein and thus may be elevated by an underlying inflammatory disease. Mile ferritin measurement is fully standardised in human medicine, a few veterinary haematology laboratories have developed ELISA-based assays for canine ferritin, e.g. Kansas State University, Veterinary Diagnostic Laboratory (http://www.ksvdl.org/laboratories/comparative-hematology/—accessed Sep. 10, 2019 and Jul. 6, 2021), but no standardised reference interval for canine serum ferritin exists.

In a healthy human, for instance, a normal ferritin blood serum level, sometimes referred to as the reference interval, is usually between 15 to 300 ng/mL in adult human males and usually between 12 to 250 ng/mL in adult human females. In an iron-deficient subject, however, serum ferritin levels are typically markedly reduced as the amount of iron available to be bound by ferritin and stored in the liver is decreased, which occurs as the body loses its ability to absorb and store iron.

The term “serum ferritin” (s-ferritin) as used herein refers to the level of ferritin in blood serum as measured using a species-specific two-site immunoenzymatic (“sandwich”) assay or another reliable quantitative serum ferritin assay. Ferritin is the major iron storage protein for the body. The concentration of ferritin is directly proportional to the total iron stores of the body, resulting in serum ferritin levels becoming a common diagnostic tool in the evaluation of iron status. Subjects with iron-deficiency anaemia have serum ferritin levels approximately one tenth of normal subjects. Ferritin levels also provide a sensitive means of detecting iron deficiency at an early stage.

In some embodiments, subjects undergoing therapy according to the methods disclosed herein experience an increase in serum ferritin levels. In some embodiments, the present disclosure provides methods of increasing serum ferritin in a subject in need thereof, the methods comprising administering an iron complex compound to the subject, wherein the iron complex compound provides an increase in serum ferritin.

In some embodiments, the iron complex compound provides a mean increase in serum ferritin that is greater than 100 ng/mL, greater than 110 ng/mL, greater than 120 ng/mL, greater than 130 ng/mL, greater than 140 ng/mL, greater than 150 ng/mL, greater than 160 ng/mL, greater than 170 ng/mL, greater than 180 ng/mL, greater than 190 ng/mL, or greater than 200 ng/mL at 4 or 8 weeks after treatment.

In some embodiments, the iron complex compound provides a mean increase in serum ferritin that is selected from less than 400 ng/mL, less than 390 ng/mL, less than 380 ng/mL, less than 370 ng/mL, less than 360 ng/mL, less than 350 ng/mL, less than 340 ng/mL, less than 330 ng/mL, less than 320 ng/mL, less than 310 ng/mL, less than 300 ng/mL, less than 290 ng/mL, less than 280 ng/mL, less than 270 ng/mL, less than 260 ng/mL, or less than 250 ng/mL at 4 or 8 weeks after treatment.

In some embodiments, the iron complex compound provides a mean increase in serum ferritin of 100 to 400 ng/mL, 100 to 375 ng/mL, 100 to 350 ng/mL, 100 to 325 ng/mL, 100 to 300 ng/mL, 100 to 275 ng/mL, or 150 to 300 ng/mL at 4 or 8 weeks after treatment.

In some embodiments, the iron complex compound provides a mean increase in serum ferritin that is greater than 200 ng/mL, greater than 230 ng/mL, greater than 260 ng/mL, greater than 290 ng/mL, greater than 320 ng/mL, greater than 350 ng/mL, greater than 380 ng/mL, greater than 410 ng/mL, or greater than 440 ng/mL at 1 week after treatment.

In some embodiments, the iron complex compound provides a mean increase in serum ferritin that is selected from less than 600 ng/mL, less than 590 ng/mL, less than 580 ng/mL, less than 570 ng/mL, less than 560 ng/mL, less than 550 ng/mL, less than 540 ng/mL, less than 530 ng/mL, less than 520 ng/mL, less than 510 ng/mL, less than 500 ng/mL, less than 490 ng/mL, less than 480 ng/mL, less than 470 ng/mL, less than 460 ng/mL, or less than 450 ng/mL at 1 week after treatment.

In some embodiments, the iron complex compound provides a mean increase in serum ferritin of 200 to 600 ng/mL, 250 to 600 ng/mL, 300 to 600 ng/mL, 350 to 600 ng/mL, or 400 to 600 ng/mL at 1 week after treatment.

The mean increases in serum ferritin apply particularly to the treatment of humans. Similar values may be applicable to companion animals, particularly canines and felines, most preferably dogs.

In addition to stored iron, a small amount of iron, circulates through the blood plasma bound to a protein called transferrin. Therefore, serum iron (s-iron) levels can be represented by the amount of iron circulating in the blood that is bound to the protein transferrin. Transferrin is a glycoprotein produced by the liver that can bind one or two ferric iron (iron(III) or Fe3+) ions. It is the most prevalent and dynamic carrier of iron in the blood, and therefore is an essential component of the body's ability to transport stored iron for use throughout the body. Transferrin saturation (or TSAT) is measured as a percentage and is calculated as the ratio of serum iron and total iron-binding capacity, multiplied by 100. This value tells a clinician how much serum iron is actually bound to the total amount of transferrin that is available to bind iron. For instance, a TSAT value of 35% means that 35% of the available iron-binding sites of transferrin in a blood sample are occupied by iron. In a healthy dog, for instance, typical TSAT values are approximately 15-50%. In an iron-deficient subject, however, TSAT values are typically markedly reduced as the amount of iron available to be bound by transferrin is decreased, which occurs as the body loses its ability to absorb and store iron. In some embodiments, the TSAT value of an iron-deficient subject is below 20% and/or the ferritin concentration is <100 μg/L.

In some embodiments, subjects undergoing therapy according to the methods disclosed herein experience an increase in TSAT values. In some embodiments, the present disclosure provides methods of increasing TSAT in a subject in need thereof, the methods comprising administering an iron complex compound to the subject, wherein the iron complex compound provides an increase in TSAT in the subject.

In some embodiments, the iron complex compound provides a mean increase in TSAT that is greater than 1%, greater than 1.5%, greater than 2%, or greater than 2.5% at 4 or 8 weeks after treatment.

In some embodiments, the iron complex compound provides a mean increase in TSAT that is less than 5%, less than 4%, or less than 3% at 4 or 8 weeks after treatment.

In some embodiments, the iron complex compound provides a mean increase in TSAT of 1 to 5%, 1.5 to 4%, or 2 to 3% at 4 or 8 weeks after treatment.

In some embodiments, the iron complex compound provides a mean increase in TSAT that is greater than 5%, greater than 6%, or greater than 7% at 1 week after treatment.

In some embodiments, the iron complex compound provides a mean increase in TSAT that is less than 20%, less than 19%, less than 18%, less than 17%, less than 16%, or less than 15% at 1 week after treatment.

In some embodiments, the iron complex compound provides a mean increase in TSAT of 5 to 20%, or 5 to 15% at 1 week after treatment.

In situations of doubt, where the collected information cannot completely rule out causes of anaemia other than iron deficiency, a bone marrow aspirate may be considered. An absence of stainable iron in the bone marrow may indicate a lack of iron for erythropoiesis in dogs.

Companion animals that are particularly suited for therapy according to the present invention are those having one or more of the following:
a haemoglobin concentration (Hb) of less than 11 g/dL;
a haematocrit (Hct/PCV) of less than 35%;
a mean corpuscular volume (MCV) of less than 60 fL;
a reticulocyte haemoglobin content (CHr)/reticulocyte haemoglobin equivalent (RET-He) of 20 pg or less; and/or
a mean corpuscular haemoglobin concentration (MCHC) of 30 g/dL or less.

In some embodiments, subjects undergoing therapy according to the methods disclosed herein may experience an improvement in iron deficiency and/or iron deficiency anaemia because Hb is raised and/or maintained above a threshold level. In some embodiments, a method of therapy of iron deficiency and/or iron deficiency anaemia is disclosed, the method comprising administering an iron complex compound to the subject, wherein the iron complex compound provides one or more of the following:
a haemoglobin concentration (Hb) of 11 g/dL or more;
a haematocrit (Hct/PCV) of 35% or more;
a mean corpuscular volume (MCV) of 60 fL or more;
a reticulocyte haemoglobin content (CHr)/reticulocyte haemoglobin equivalent (RET-He) of more than 20 pg; and/or
a mean corpuscular haemoglobin concentration (MCHC) of more than 30 g/dL.

The present disclosure provides methods of improving one or more iron storage parameters in a subject in need thereof. The at least one iron storage parameter may be selected from serum ferritin levels, transferrin saturation (TSAT), haemoglobin concentration, haematocrit, total iron-binding capacity, iron absorption levels, serum iron levels, liver iron levels, spleen iron levels, and combinations thereof.

In one embodiment, the at least one iron storage parameter is haemoglobin concentration, and improving comprises increasing the haemoglobin concentration of the subject. In other embodiments, the at least one iron storage parameter is transferrin saturation, and improving comprises increasing the transferrin saturation of the subject. In yet other embodiments, the at least one iron storage parameter is serum ferritin levels, and improving comprises increasing the serum ferritin levels of the subject.

### Iron Complex Compounds

Described herein are therapeutic methods, i.e., therapy of iron deficiency which comprise administering an iron complex compound, and combinations of an iron complex compound with additional drugs, wherein the iron complex compound has certain properties and thus exerts certain effects in subjects undergoing therapy. The methods of the invention are thus applicable to complexes that share said properties. For instance, the iron complex compound should be relatively stable; have good absorption properties; and show low urinary excretion.

Unless further specified, the term “iron complex compound” as used herein refers to any complex of iron ions or iron particles comprising Fe3+ and/or Fe2+ and one or more ligands. The iron atoms are bound in a coordination complex through ionic and coordinate covalent bonds with the ligand(s) or as part of a poly-nuclear iron ligand nanomolecule, preferably an iron carbohydrate nanomolecule.

### Ligands

Expediently, the ligands and salts used in the iron complex compounds of the invention as well as the carriers and other ingredients of the compositions thereof are physiologically acceptable. The term “physiologically acceptable” as used herein, means that the ligand, salt, carrier or other ingredient does not cause acute toxicity when a therapeutically effective amount of the iron complex compound or the composition comprising the ligand, salt, carrier or other ingredient is administered to a subject.

### Carbohydrates

According to one group of embodiments, the ligand in the iron complex compound is a carbohydrate.

Unless further specified, the term “carbohydrate” as used herein includes carbohydrates which are reduced, oxidised, derivatised or a combination thereof as described herein. In particular, carbohydrates can be derivatised, for example, by the formation of ethers, amides, esters and amines with the hydroxyl groups of the carbohydrates or by the conversion of aldehyde groups of the carbohydrates to glycolic groups so as to form heptonic acids. The term “carbohydrate” as used herein is thus not limited to compounds having the empirical formula Cₘ(H₂O)ₙ, wherein m and n are integers which may be the same or different from each other.

Carbohydrates which may be used as ligands in iron carbohydrate complexes of the present invention include, for example, monosaccharides; disaccharides, e.g. sucrose, maltose or isomaltose; oligosaccharides and polysaccharides, e.g. maltodextrin, polyglucose, dextran, oligomaltose, oligoisomaltose; sugar alcohols, e.g. sorbitol and mannitol; sugar acids and salts thereof, e.g. gluconic acid, gluconate, dextran glucoheptonic acid, dextrin glucoheptonic acid, dextran glucoheptonate and dextrin glucoheptonate, as well as reduced and/or oxidised and/or derivatised variants thereof, e.g. carboxymaltose, polyglucose sorbitol carboxymethyl ether, hydrogenated dextran, oxidised dextran, carboxyalkylated oligo- and polysaccharides, oxidised oligo- and polysaccharides, hydrogenated dextrin, oxidised dextrin, hydrogenated oligomaltose, hydrogenated oligoisomaltose, hydrogenated oligomaltose, hydroxyethyl starch, hydroxyethyl starch carrying heptonic acid moieties, or a mixture of two or more thereof. When oligo- and polysaccharides are used, these typically comprise mixtures of oligo- and polysaccharides having varying chain lengths. Therefore, these oligo- and polysaccharides can conveniently be characterised by weight or number average molecular weights and the distribution of these molecules across a range of molecular weights. For the sake of simplicity, reference to an oligo- or polysaccharide is meant to refer to such mixtures.

The term “oligosaccharide” as used herein generally refers to a carbohydrate, or a reduced and/or oxidised and/or derivatised variant thereof, having a small number, typically 3-10, monosaccharide units, or to a mixture of two or more carbohydrates, or reduced and/or oxidised and/or derivatised variants thereof, wherein the majority (e.g., at least 60%, at least 70%, or at least 80%) of the molecules have a small number, typically 3-10, of monosaccharide units.

The term “monomer saccharide” as used herein refers to a monosaccharide or a reduced and/or oxidised and/or derivatised variant thereof, or to a mixture of two or more monosaccharides and/or variants thereof.

The term “dimer saccharide” as used herein refers to a carbohydrate having two monosaccharide units (such as a disaccharide) or a reduced and/or oxidised and/or derivatised variant thereof, or to a mixture of two or more carbohydrates, or reduced and/or oxidised and/or derivatised variants thereof, wherein the molecules have two monosaccharide units.

Sugar alcohols are mono- or disaccharide derivatives wherein the aldehyde group is converted to a hydroxyl group.

Sugar acids are monosaccharide derivatives which carry a carboxyl group. The carboxyl group can be obtained by, for example, oxidizing the aldehyde group of an aldose so as to form an aldonic acid, oxidizing the 1-hydroxyl group of a 2-ketose so as to form an α-ketoacid (ulosonic acid), oxidizing the terminal hydroxyl group of an aldose or ketose so as to obtain an uronic acid, or oxidizing both ends of an aldose so as to obtain an aldaric acid.

Preferably, the content of reducing aldehyde groups in the carbohydrate is at least partially reduced. This can be achieved by hydrogenation, oxidation, glycosylation, or a combination thereof. Iron carbohydrate complex compounds comprising carbohydrates which are hydrogenated and/or oxidised can be prepared as described, for example, in WO 99/48533 A1; WO 2010/108493 A1 or WO 2019/048674 A1, all of which is incorporated by reference. The amount of reducing carbohydrate can be determined using Somogyi's reagent.

Specifically, the aldehyde groups can be converted into hydroxyl groups by hydrogenation, for example by reacting the carbohydrate with a reducing agent, such as sodium borohydride in aqueous solution, or with hydrogen in the presence of a hydrogenation catalyst, such as Pt or Pd.

Alternatively or additionally to hydrogenation, aldehyde groups can be oxidised, for example by oxidation of the carbohydrate using an aqueous solution of hypochlorite, chlorite or hypobromite at a pH within the akaline range, e.g. within the range of from pH 8 to pH 12, in particular from pH 9 to pH 11. Suitable hypochlorites include, for example, alkali metal hypochlorites such as sodium hypochlorite, and same applies to chlorites and hypobromites. The aqueous solution of hypochlorite, chlorite or hypobromite can have a concentration of, for example, at least 13 wt-%, in particular in the range of from 13 to 16 wt-%, calculated as active chlorine. The oxidation reaction can be performed at temperatures in the range of, for example, from 15 to 40° C., preferably from 25 to 35° C. Reaction times are, for example, in the range of from 10 min to 4 hours, such as from 1 to 1.5 hours. The addition of catalytic amounts of bromine ions, e.g. in the form of alkali metal bromides such as sodium bromide, can further the oxidation reaction but is not mandatory.

The aldehyde groups of the carbohydrate can be converted by both hydrogenation and oxidation. This can be achieved, for example, in that the carbohydrate is first hydrogenated to convert part of the aldehyde groups into hydroxyl groups, and then substantially all of the remaining aldehyde groups are oxidised to carboxyl groups. Where the carbohydrate is a polysaccharide such as dextran, the average molecular weight of the iron carbohydrate complex formed therewith can be influenced by adjusting the ration of hydrogenated aldehyde groups to oxidised aldehyde groups. To obtain a stable product, the amount of reducing groups in the carbohydrate (e.g., dextran) before oxidation does not exceed 15 wt-%.

The carbohydrates, including reduced and/or oxidised carbohydrates, can be derivatised by formation of, for example, ethers, amides, esters and amines with the hydroxyl groups of the carbohydrates. In a particular embodiment, the carbohydrate is derivatised by formation of carboxyalkyl ether, in particular carboxymethyl ether, with a hydroxyl group of the carbohydrate. The use of carboxymethylated carbohydrate in a product such as an iron carbohydrate complex compound of the invention may reduce the toxicity of the product when administered parenterally to a subject compared to a product comprising a corresponding non-carboxylated carbohydrate.

In preferred embodiments, the carbohydrate is carboxymaltose, polyglucose sorbitol carboxymethyl ether, dextran, hydrogenated dextran, dextran glucoheptonic acid, dextran glucoheptonate, dextrin, hydrogenated dextrin, dextrin glucoheptonic acid, dextrin glucoheptonate, oligoisomaltose, hydrogenated oligoisomaltose, hydroxyethyl starch, hydrogenated hydroxyethyl starch, hydroxyethyl starch carrying heptonic acid moieties, hydroxypropyl starch, hydrogenated hydroxypropyl starch, hydroxypropyl starch carrying heptonic acid moieties, or a mixture of two or more thereof.

Such carbohydrates will typically have a weight average molecular weight (M_{w}) of from 500 to 80,000 Da, such as from 800 to 40,000 Da or from 800 to 10,000 Da and in particular from 800 to 3,000 Da. In particular embodiments, the carbohydrate is a polysaccharide or oligosaccharide or mixture thereof having a weight average molecular weight (M_{w}) of from 500 to 7,000 Da, such as from 500 to 3,000 Da, from 700 to 1,400 Da and in particular of from 850 to 1,150 Da, e.g., of about 1,000 Da, or from 1,150 to 1,350 Da, e.g., of about 1,250 Da.

The amount of dimer (disaccharide) in carbohydrate preparations which are (optionally reduced and/or oxidised and/or derivatised) oligosaccharide or polysaccharide preparations is considered to be a key factor with regard to the physiological iron release rate from the iron carbohydrate complex compounds prepared therefrom. See WO 2010/108493 A1. Therefore, where the carbohydrate is an (optionally reduced and/or oxidised and/or derivatised) oligosaccharide or polysaccharide preparation, such as the hydrogenated poly-/oligosaccharides disclosed herein, the content of dimer saccharides in said preparation is preferably 2.9 wt-% or less, in particular 2.5 wt-% or less, and especially 2.3 wt-% or less, based on the total weight of the carbohydrate. It is also preferred that the content of monomer saccharide in the carbohydrate preparation is 0.5 wt-% or less, based on the total weight of the carbohydrate. This reduces the risk of toxic effects caused by free iron ions released from the compounds after parenteral administration.

Particularly preferred carbohydrate ligands are described below.

### Oligoisomaltose

In particularly preferred embodiments, the carbohydrate is oligoisomaltose or, even more preferably, hydrogenated oligoisomaltose (i.e., oligoisomaltoside).

In particular embodiments, the oligoisomaltos(id)e has a weight average molecular weight (M_{w}) of from 700 to 1,500 Da. Oligoisomaltos(id)e having a weight average molecular weight (M_{w}) of from 850 to 1,150 Da; preferably 950 to 1,050 Da, most preferably from 975 to 1025 Da, e.g., of about 1000 Da, represents one particular embodiment. Oligoisomaltos(id)e having a weight average molecular weight (M_{w}) of from 1,150 to 1,350 Da; preferably from 1,200 to 1,300 Da, most preferably from 1,225 to 1275 Da; e.g., of about 1250 Da (also referred to herein as “octasaccharide”), represents another particular embodiment. For the oligoisomaltos(id)e having a weight average molecular weight (M_{w}) of from 850 to 1,150 Da it is preferred if the fraction with more than 9 monosaccharide units is less than 30%, preferably less than 25%, most preferably less than 20%; e.g., 5% to 15%, by weight of the oligosaccharide. For the oligoisomaltos(id)e having a weight average molecular weight (M_{w}) of from 1,150 to 1,350 Da it is preferred if the fraction with more than 9 monosaccharide units is less than 40%, preferably less than 35%, most preferably less than 30%; e.g., 20 to 30%, by weight of the oligosaccharide. According to another aspect, the content of monomer and dimer (the fraction with less than 3 monosaccharide units) is less than 10.0%, preferably less than 3.0%, most preferably less than 1.0%; e.g., 0.1 to 0.5%, by weight of the oligosaccharide.

Oligoisomaltos(id)es wherein a major proportion (such as at least 40% or preferably at least 50%, e.g., from 40 to 70% or from 50 to 70% by weight) of the molecules has 3-6 monosaccharide units, represent one preferred embodiment. This applies in particular to those oligoisomaltos(id)e having a weight average molecular weight (M_{w}) of from 850 to 1,150. Accordingly, in preferred embodiments of the invention, the ligand is an oligoisomaltos(id)e wherein a major proportion (such as at least 40% or preferably at least 50%, e.g., from 40 to 70% or from 50 to 70% by weight) of the optionally hydrogenated oligoisomaltose molecules have 3-6 monosaccharide units. More specifically, said proportion of molecules having 3-6 monosaccharide units is higher than the proportion of molecules having 6-10 monosaccharide units. An example of such oligosaccharide is isomaltoside 1000 (INN name: derisomaltose).

Oligoisomaltos(id)es wherein a major proportion (such as at least 40% or preferably at least 45%, e.g., from 40 to 60% or from 45 to 55% by weight) of the molecules has 6-10 monosaccharide units (also referred to herein as “octasaccharide”), represent another preferred embodiment. This applies in particular to those oligoisomaltos(id)e having a weight average molecular weight (M_{w}) of from 1,150 to 1,350. Accordingly, in preferred embodiments of the invention, the ligand is an oligoisomaltos(id)e wherein a major proportion (such as at least 40%, e.g., from 40 to 60% by weight) of the optionally hydrogenated oligoisomaltose molecules have 6-10 monosaccharide units. More specifically, said proportion of molecules having 6-10 monosaccharide units is higher by weight than the proportion of molecules having 3-6 monosaccharide units. An example of such oligosaccharide is the octasaccharide disclosed herein.

The oligoisomaltos(id)es of the invention are preferably hydrogenated oligoisomaltoses (oligoisomaltosides). Typically, the amount of reducing sugar in such hydrogenated oligoisomaltose (oligoisomaltoside) is 2.5% or less, preferably 1.0% or less, most preferably 0.5% or less; e.g. about 0.3%, by weight of the oligosaccharide. Prior to hydrogenation, the amount of reducing sugar in the oligoisomaltoses is at least 10% and usually at least 15%, by weight of the oligosaccharide. However, the amount of reducing sugar also depends on the molecular weight distribution of the carbohydrate chains. A shorter chain contributes relatively high amounts of reducing sugar while a longer contributes less. Accordingly, it is a particular aspect of the invention that the amount of reducing sugar in the oligoisomaltoses is less than 35%, preferably no more than 30%; e.g., in the range of 10% to 30%, preferably in the range of 15 to 25%, by weight of the oligosaccharide.

### Gluconic Acid Derivatives

Another particular carbohydrate ligand for use in this invention are gluconic acid derivative of carbohydrates such as dextran or dextrin. Examples include bepectate or dextran glucoheptonic acid. The term “bepectate” as used herein refer to a hydroxyethyl-amylopectin (starch) derivative. Bepectate has also been referred to as polyglucoferron. Bepectate is disclosed, for instance, in WO 2012/175608 A1, all of which is incorporated by reference. Such hydroxyethyl-amylopectin (starch) derivative might carry a number of heptonic acid residues per molecule, depending on the number of terminal glucosyl residues being present in the starch molecule. This heptonic acid residue increases the hydrophilicity of the hydroxyethyl starch and increases the stability of complexes formed by this hydroxyethyl starch with ligands, like for example metal ions such as iron ions. Speaking more generally, hydroxyethyl starch (HES) is a starch in which some of the hydroxyl groups of the single glucosyl residues are substituted by a hydroxyethyl residue. The modification by the heptonic acid residue takes place by converting the terminal glucosyl residue of the hydroxyethyl starch into a heptonic acid residue. Preferably, the hydroxyethyl starch used in the method has a weight average molecular weight (Mw) of less than 200,000 g/mol, in particular of less than 130,000 g/mol, in particular of less than 100,000 g/mol, in particular of less than 90,000 g/mol, in particular of less than 80000 g/mol and very particular of less than 75,000 g/mol. A very well suited molecular weight is in the range of 55,000 g/mol to 85,000 g/mol. Such a hydroxyethyl starch has a comparatively lower molecular weight than (non-modified) hydroxyethyl starches used in the medical field at present. A suited method for determining the molecular weight of the hydroxyethyl starch is size exclusion chromatography (SEC). In a preferred embodiment, the hydroxyethyl starch has an average degree of molar substitution of 0.4 to 0.6, in particular of 0.45 to 0.55. An average degree of molar substitution of around 0.50 is particularly preferred. The average degree of molar substitution is a measure for the amount of hydroxyl groups being substituted by a hydroxyethyl residue per glucosyl residue. Since each glucose unit (or glucosyl residue) bears three hydroxyl groups, the average degree of molar substitution can be three at the maximum. An average degree of molar substitution of 0.5 indicates that (on an average or statistic basis) in each second glucosyl residue one hydroxyl group is substituted by a hydroxyethyl residue. In a preferred embodiment, the hydroxyethyl starch has a weight average molecular weight (Mw) of 55,000 to 85,000 g/mol, preferably around 70,000 g/mol, and an average degree of molar substitution of 0.45 to 0.55, in particular around 0.50. Such a hydroxyethyl starch with a molecular weight of 70,000 g/mol±15,000 g/mol and an average degree of molar substitution of 0.5±0.05 can also be referred to as HES 70/0.5.

Dextran glucoheptonic acid, dextran glucoheptonate, dextrin glucoheptonic acid, and dextrin glucoheptonate are further examples suitable carbohydrate ligands where a saccharide such as dextran or dextrin is modified so as to carry heptonic acid residues.

### Polymeric Ligands

According to another group of embodiments, the ligand is a ligand suitable for ligand-substituted oxo-hydroxy iron complex compounds. Suitable ligands include, for example, carboxylic acids, such as adipic acid, glutaric acid, tartaric acid, malic acid, succinic acid, aspartic acid, pimelic acid, citric acid, gluconic acid, lactic acid and benzoic acid; food additives such as maltol, ethyl maltol and vanillin; anions with ligand properties such as bicarbonate, sulphate and phosphate; mineral ligands such as silicate, borate, molybdate and selenate; amino acids, in particular proteinogenic amino acids, such as tryptophan, glutamine, proline, valine and histidine; and nutrient-based ligands such as folate, ascorbate, pyridoxine and niacin; as well as a mixtures of two or more thereof. A particular example of suitable polymeric ligands are the biocompatible polyethylene glycol-based polymers described in U.S. Pat. No. 8,741,615 B2, i.e., a biocompatible polymer of general formula (1),

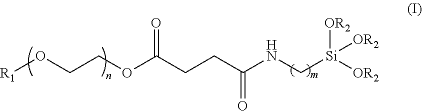

wherein R₁ is alkyl, aryl, carboxyl, or amino, R₂ is alkyl or aryl, n is an integer from 5 to 1000, and m is an integer from 1 to 10. Suitable alkyl groups for R₁ and R₂ include C₁-C₂₀ straight chain or branched alkyl groups. In one embodiment, each of R₁ and R₂ independently, is a C₁-C₆ straight chain or branched alkyl such as methyl, ethyl, propyl, isopropyl, n-butyl, sec-butyl, tert-butyl, n-pentyl, isopentyl, tert-pentyl, n-hexyl, and isohexyl. Suitable aryl groups for R₁ and R₂ include C₆-C₁₂ substituted or unsubstituted aryl groups such as phenyl, biphenyl, and naphthyl, and examples of substituents thereof include hydroxyl, haloalkyl, alkoxyl, cyano, nitro, amino, or alkylamino. The number of methylene units m is preferably an integer from 1 to 10. The number of oxyethylene units n is preferably an integer from 5 to 1000, equivalent to a molecular weight of 200-50000 g/mole of the PEG. In one embodiment, m is about 3, and n is about 15.

The biocompatible polymer is useful in that it can chemically modify the surface of iron oxide nanoparticles to give a biocompatible magnetic material comprising a magnetic nanoparticle and the biocompatible polymer.

### Preparation of Carbohydrates

The manufacture of most of the carbohydrates disclosed herein requires their preparation from readily available carbohydrates. Common starting materials are dextrans and dextrins, i.e., polyglucoses with predominantly α-1,6- or α-1,4-linked glucose units, respectively. Because the dextrans and dextrins that are used as starting materials are typically high molecular weight polysaccharides, these usually need to be hydrolysed and the resulting hydrolysates fractionated in order to adjust the molecular weight of the desired carbohydrates.

A typical process for producing oligoisomaltos(id)es of the invention comprises the following steps:
(a) hydrolysis of dextran to obtain a hydrolysate;
(b) fractionation of the hydrolysate to obtain the oligoisomaltose; and optionally
(c) hydrogenation of the oligoisomaltose to obtain the oligoisomaltoside.

Further optional steps are:
Purification, e.g., by diafiltration, to reduce the level of mono- and disaccharide in the oligoisomaltoside.
Purification, e.g., by ion exchange, to obtain a purified oligoisomaltose or oligoisomaltoside.

For instance, oligoisomaltos(id)es such as the octasaccharide of the invention can be manufactured from dextran fractions that are combined and fractionated by ultrafiltration. The dextran fractions can be produced from intermediate dextran having a weight-average molecular weight (M_{w}) in the range of 500 to 2000 kDa, which are hydrolysed to have a Mw in the range of 20,000 to 70,000 Da. In one or more steps, the starting material can be hydrolysed to a lower molecular weight, fractionated and filtrated until the desired molecular weight distribution is achieved. The resulting oligoisomaltose can then be hydrogenated to yield oligoisomaltoside. Purification by diafiltration can help to reduce the level of mono- and disaccharide and the resulting product can be further purified, for instance, by ion exchange. For instance, low amounts of mono- and disaccharide can be achieved by removing said smaller saccharide molecules from a carbohydrate preparation by membrane filtration, for examples using membranes having cut-off values in the range of 340-800 Da. The concentration of mono- and disaccharide in the fractions obtained by the purification method can be monitored by gel permeation chromatography.

Bepectate's manufacture is disclosed, for instance, in WO 2012/175608 A1. Briefly, hydroxyethyl starch is dissolved in water. Then, the pH value is adjusted to a value of 8.0 to 10.0. Afterwards, a cyanide compound is added to the hydroxyethyl starch solution. Then the solution is heated to a temperature of 80 to 99° C. and kept at this temperature for a first time period. Finally, the pH value is adjusted to a value of 2.0 to 4.0 and the solution is brought to a temperature of 50 to 90° C. and kept at this temperature for a second time period. The manufacture of gluconic acid derivatives of dextran and dextrin is disclosed, for instance, in U.S. Pat. No. 3,639,588. The manufacture of polyethylene glycol-based polymers is disclosed, for instance, in U.S. Pat. No. 8,741,615.

### Iron Preparation

Iron preparations that can be used for making the iron complex compounds comprise iron in a form that is selected from a water-soluble iron salt, an iron hydroxide and an iron oxide-hydroxide. The iron preparation may contain a mixture of two or more of these iron forms.

In a particular embodiment, the iron preparation comprises a water-soluble iron salt, for example an iron bromide, sulfate or chloride, in particular ferric chloride (FeCl₃), ferrous chloride (FeCl₂) or a mixture thereof. Expediently, the water-soluble iron salt is a physiological acceptable salt.

In a further particular embodiment, the iron preparation comprises iron hydroxide, for example ferric hydroxide (Fe(OH)₃), ferrous hydroxide (Fe(OH)₂) or a mixture thereof.

In a further particular embodiment, the iron preparation comprises iron oxide-hydroxide. Iron oxide-hydroxides may also be termed iron oxy-hydroxides. Iron oxide-hydroxides are compounds which consist of one or more than one iron ion, one or more than one oxo group, and one or more than one hydroxyl group. Particular iron oxide-hydroxides include, e.g., ferric oxide-hydroxides which occur in anhydrous (FeO(OH)) forms and hydrated (FeO(OH)·nH₂O) forms such as, e.g., ferric oxide-hydroxide monohydrate (FeO(OH)·H₂O). Iron oxide-hydroxides can be prepared for example from aqueous iron(II) salt solutions by hydrolysis and precipitation as described, e.g., in Roempp lexicon Chemie, 10. Auflage, 1997. Iron oxide-hydroxides can be present in different polymorphic forms. For example, polymorphs of FeO(OH) include α-FeO(OH) (goethite), α-FeO(OH) (akagnéite), γ-FeO(OH) (lepidocrocite) and δ-FeO(OH) (feroxyhyte).

According to a particular embodiment, an iron preparation that is low in non-iron metal impurities is used. Appropriate levels for such non-iron metal impurities are described in WO 2019/048674 A1. Such a preparation can be obtained
(a) from iron pentacarbonyl; or
(b) by recrystallization of an iron salt from an aqueous solution thereof; or
(c) by extracting an aqueous iron salt solution with an organic solvent; or
(d) from iron precipitated at an anode during electrolysis of an aqueous iron salt solution; or
(e) by contacting an aqueous iron salt solution with a base so as to form a precipitate of iron hydroxide and separating the precipitate from the liquid by filtration or centrifugation; or
(f) by distillation of ferric chloride from a mixture comprising ferric chloride and non-volatile impurities.

According to a preferred embodiment, the iron preparation is obtained by a process wherein an aqueous iron salt solution (e.g., an aqueous iron salt solution obtained during the processing of an iron-containing nickel ore for nickel production) is extracted with an organic solvent.

According to a particularly preferred embodiment, the iron preparation used in the process of the invention is prepared from iron pentacarbonyl.

The generation of an iron preparation as described herein:
(a) from iron pentacarbonyl, or
(b) by recrystallization of an iron salt from an aqueous solution thereof, or
(c) by extracting an aqueous iron salt solution with an organic solvent, or
(d) from iron precipitated at an anode during electrolysis of an aqueous iron salt solution, or
(e) by contacting an aqueous iron salt solution with a base so as to form a precipitate of iron hydroxide and separating the precipitate from the liquid by filtration or centrifugation, or
(f) by distillation of ferric chloride from a mixture comprising ferric chloride and non-volatile impurities can, but is not required to, be a step of the process of the present invention.

Methods for preparing a water-soluble iron salt, iron hydroxide or iron oxide-hydroxide from iron pentacarbonyl are known in the art. For example, in a first step, iron pentacarbonyl can be decomposed to form iron (so-called carbonyl iron) at an elevated temperature (e.g. 200° C. or more), optionally in the presence of a catalyst such as H₂, NO, PF₃, PH₃, NH₃ and/or I₂, as described for example in U.S. Pat. No. 4,056,386. The iron can be reacted with (preferably an excess of) hydrochloric acid so as to obtain FeCl₂. FeCl₂ can be reacted with hydrochloric acid and (preferably a slight deficit of) sodium chlorate so as to obtain FeCl₃. FeCl₂ can be reacted with hydrochloric acid and oxidised using, for example, hydrogen peroxide so as to form FeCl₃. This reaction can be used to oxidize FeCl₂ remaining from the reaction with hydrochloric acid and sodium chlorate so as to achieve a more complete conversion of FeCl₂ to FeCb₃. Also chlorine (Cl₂; gas) can be used as oxidation agent.

Carbonyl iron can be prepared, for example, from iron pentacarbonyl which can be prepared, for example, by directing carbon monoxide onto hot iron (e.g., as hot as about 200° C.), preferably under high pressure (e.g., as high as 15-20 MPa). Such preparation of carbonyl iron via iron pentacarbonyl is described, for example, in French patent application no. 607.134 of Badische Anilin- & Soda-Fabrik published on Jun. 26, 1926.

Methods for preparing an iron preparation as described herein by recrystallization of an iron salt preparation from an aqueous solution thereof are known in the art. To this end, an aqueous solution of a water-soluble iron salt preparation is provided, the iron salt (e.g., ferric nitrate) is recrystallised from the solution (e.g., by reducing the temperature of the solution), the iron salt crystals are separated from the liquid, are dissolved so as to form an aqueous solution thereof and then again subjected to recrystallization and separation. The steps of dissolution, recrystallization and separation can be repeated for one or several more times so as to increase the purity, and in particular reduce the amount of non-iron metal impurities, of the iron salt preparation. According to a particular example, ferric nitrate is recrystallised from an aqueous solution thereof containing nitric acid. Specifically, ferric nitrate is dissolved in 55-65% aqueous nitric acid at 50-60° C. The solution is cooled to about 15° C. or lower temperature, where crystalline ferric nitrate precipitate is formed and can be separated from the liquid. Said steps of dissolution, recrystallization and separation can be repeated for one or several more times.

Methods for preparing an iron preparation as described herein by extracting an aqueous iron salt solution with an organic solvent are known in the art. To this end, an aqueous ferric chloride solution can be treated with an organic solvent so as to selectively dissolve the ferric chloride in the organic solvent (extraction), then the selectively dissolved ferric chloride can be recovered by stripping the organic solvent from the ferric chloride. Exemplary organic solvents include alcohols having 4-20 carbon atoms, in particular alcohols having 6-10 carbon atoms, such as n-octanol, and organic solutions of amine salts such as tri-n-laurylamine hydrochloride in toluene. The presence of hydrochloric acid in the aqueous ferric chloride solution can improve extraction efficiency. It is advantageous to increase the concentration of ferric chloride in the aqueous starting solution by partial evaporation before adding the organic solvent, in particular to a concentration in the range of 280-850 g/l ferric chloride. The purification cycle of evaporation and solvent extraction can be repeated until the desired purity of the ferric chloride preparation is obtained. Aqueous solutions of ferrous chloride can also be purified if the ferrous chloride is first converted to ferric chloride by oxidation with chlorine. Specific methods for extraction of iron salts with organic solvents are described, e.g., in CA 2318 823 A1 and Müller et al. (“Liquid-liquid extraction of ferric chloride by tri-n-laurylamine hydrochloride”, EUR 2245.e, Euratom report, Transplutonium Elements Program, Euratom Contract No. 003-61-2 TPUB, Presses Academiques Europeennes, Brussels, 1965).

Methods for electrolysis of aqueous iron salt solutions wherein iron is precipitated at an anode are known in the art. See, for example, Cain et al. (“Preparation of pure iron and iron-carbon alloy” in Bulletin of the Bureau of Standards, Vol. 13, 1916) and Mostad et al. (Hydrometallurgy, 2008, 90, 213-220). Suitable iron solutions for electrolysis include iron chloride solutions, iron sulfate solutions and solutions containing both iron chloride and iron sulfate. The solution is typically neutral or acidic.

An iron preparation as described herein can further be obtained by contacting an aqueous iron salt solution with a base so as to form a precipitate of iron hydroxide and separating the precipitate from the liquid by filtration or centrifugation. Suitable bases for precipitation of iron hydroxides include sodium hydroxide or sodium carbonate. Alternatively, sodium bicarbonate can be used. Methods for separating such precipitate from the remaining solution by filtration or centrifugation are known in the art.

An iron preparation that is low in non-iron metal impurities, such as the iron preparation used in the process of the invention, can also be prepared by distillation of a mixture comprising ferric chloride and non-volatile impurities. For distillation, the mixture is subjected to a temperature and a pressure which are chosen such that at the selected pressure and temperature the mixture is at about its boiling point. At those conditions, the mixture separates into a vapor phase and a slurry of non-volatile impurities in liquid ferric chloride. The vapor is substantially pure ferric chloride that can be recovered by separating the vapor from the slurry. According to particular embodiments, a temperature at about the boiling point of the mixture means a temperature that is within 10° C. of said boiling point and preferably is at said boiling point. Distillation can be performed, e.g., at a temperature in the range of from 300° C. to 700° C. and a pressure in the range of from 0.1 to 5.1 MPa, preferably in the range of from 0.2 to 0.4 MPa, wherein at the selected pressure and temperature the mixture is at about its boiling point.

During distillation, settlement of non-volatile solids in the slurry can be prevented by agitating the slurry mechanically (e.g., by paddle stirrer or the like) or, preferably, by bubbling a gas (e.g., nitrogen, helium, chlorine or a mixture thereof) through the slurry.

After separation of the ferric chloride vapor, the remaining slurry can be recycled by heating the slurry so as to vaporize ferric chloride, separating and cooling the ferric chloride containing vapor and reintroducing it to the distillation process. Preferably, the recycling of the slurry is performed such that the amount of solids present in the slurry during distillation is below about 20 wt-%, in particular below about 12 wt-%.

The mixture comprising ferric chloride and non-volatile impurities that is introduced into the distillation process can be obtained, for example, by chlorinating iron-containing ore (e.g., a titaniferous ore such as ilmenite) so as to produce a gaseous mixture comprising ferric chloride and non-volatile impurities and cooling the gas so as to precipitate a solid mixture of ferric chloride and non-volatile impurities. Said solid mixture can then be introduced into the distillation process. Prior to separating the solid mixture of ferric chloride and non-volatile impurities from the gaseous mixture, the gaseous mixture can optionally be subjected to a temperature above the dew-point of ferric chloride so as to remove non-volatile impurities which are no longer gaseous at this temperature. The thus pre-purified gaseous mixture can then be cooled so as to precipitate a solid mixture of ferric chloride and non-volatile impurities that can be introduced into the distillation process. See, for example, U.S. Pat. No. 3,906,077, all of which is incorporated by reference.

Different methods for preparing and purifying iron preparations can be combined so as to increase the purity of the iron preparation even further. For example, iron prepared by electrolysis of an aqueous iron salt solution can be converted into a water-soluble iron salt that is then subjected to one or more cycles of
(1) dissolution so as to form an aqueous solution of the iron salt,
(2) recrystallization of the iron salt from the aqueous solution, and
(3) separation of the recrystallised iron salt from the remaining solution.

### Complexes

For iron ions to be suitable for parenteral administration, they have to be complexed with ligands so that the amount of free iron ions is low, and the iron is released in a controlled manner after being administered. Expediently, the total amount of free iron that comes with the iron complex compound prior to administration is 0.01% w/v or less and preferably less than 0.003% w/v (for an iron complex compound being presented as a 100 mg/mL solution). Put differently, the total amount of free iron relative to the total iron content is 0.1% or less and preferably less than 0.03% of free iron by weight of total iron content (for an iron complex compound being presented as a 100 mg/mL solution). This requires the iron complex compound to have a physical stability that sufficient for the complex to be processed into the final drug product and stored until it is used.

### Iron Carbohydrate Complexes

According to one group of embodiments, the iron complex compound is an iron carbohydrate complex compound, i.e., the ligand in the iron complex compound is a carbohydrate.

Iron carbohydrate complex compounds of the present invention include complexes with the carbohydrate ligands disclosed herein, for example, iron carboxymaltose, iron polyglucose sorbitol carboxymethyl ether complex, iron mannitol complex, iron dextran, iron hydrogenated dextran, iron oxidised dextran, iron carboxyalkylated reduced oligo- and polysaccharides, iron sucrose, iron gluconate, iron dextrin, iron hydrogenated dextrin, iron oxidised dextrin, iron oligomaltose, hydrogenated iron oligomaltose, iron hydrogenated oligosaccharides such as iron hydrogenated oligoisomaltose, iron hydroxyethyl starch, iron sorbitol, iron dextran glucoheptonic acid (e.g., gleptoferron) and a mixture of two or more thereof. According to particular embodiments, the iron carbohydrate complex compound of the present invention is selected from iron carboxymaltose, iron polyglucose sorbitol carboxymethyl ether complex, iron mannitol complex, iron dextran, iron hydrogenated dextran, iron sucrose, iron gluconate, iron dextrin, iron hydrogenated oligoisomaltose and a mixture of two or more thereof. In more preferred embodiments, the iron carbohydrate complex is iron hydrogenated oligoisomaltose (iron oligoisomaltoside).

The amount of iron in the iron carbohydrate complex compound of the invention, determined for dry matter, is typically in the range of from 10 to 50%, preferably 15 to 35%, most preferably 20 to 30%, e.g., from 20 to 25%, of iron by weight of the carbohydrate complex.

Accordingly, the weight ratio of elemental iron to carbohydrate in the complex is typically 10:90 to 50:50, preferably 15:85 to 45:55, most preferably 20:80 to 40:60, e.g., about 70:30.

The “apparent” peak molecular weight (Mₚ) of the iron carbohydrate complexes of the invention is typically in the range of from 800 to 800,000 Da, such as from 10,000 to 500,000 Da or from 20,000 to 400,000 Da or from 50,000 to 300,000 Da and in particular from 90,000 to 200,000 Da. The “apparent” peak molecular weight Mₚ can be determined by gel-permeation chromatography using, e.g., dextran standards. See, for example, the method described in Jahn et al., Eur J Pharm Biopharm 2011, 78, 480-491. For iron oligoisomaltos(id)e complexes disclosed herein the “apparent” peak molecular weight (Mₚ) is typically in the range of from 120,000 to 190,000 Da, in particular from 125.000 to 185.000 Da or from 130,000 to 180,000 Da. An “apparent” peak molecular weight (Mₚ) in the range of 135,000 to 175,000 Da and especially in the range from 140,000 to 155,000 Da has proven to be advantageous, especially in connection the ferric octasaccharide disclosed herein. Preferably, the “apparent” peak molecular weight (Mp) is in the range of 145,000 to 155,000 Da, especially in connection the ferric octasaccharide disclosed herein. The iron oligoisomaltos(id)es of the invention preferably have a relatively narrow molecular weight distribution with a dispersity (Mw/Mn) in the range of 1.0 to 1.5, preferably 1.05 to 1.4, more preferably 1.1 to 1.3; e.g., at about 1.2.

In some embodiments, the iron carbohydrate complexes of the invention can comprise stabilizers such as organic acids. Preferably, the organic acid is an organic hydroxy acid. Suitable examples of organic hydroxy acids are gluconic acid and citric acid. Citric acid is an expedient example. If present, the amount of citric acid is typically in the range of 3 to 20% by weight of total quantity of elemental iron.

Accordingly, iron carbohydrate complexes that are particularly suitable for use in this invention are iron oligoisomaltos(id)es, such as iron isomaltoside 1000 (INN name: ferric derisomaltose) or ferric octasaccharide, as disclosed herein. The term “iron oligoisomaltosides” as used herein refers to colloidal complexes comprising iron, e.g., as iron oxide hydroxide, and oligoisomaltoside in a matrix-like structure.

Iron oligoisomaltosides are the preferred iron carbohydrate complexes for use according to the invention. In a preferred embodiment, the iron carbohydrate complex for use in the invention comprises iron oxide hydroxide in stable association with an octasaccharide. In a preferred embodiment, the iron carbohydrate complex is ferric octasaccharide,

An example of another iron oligoisomaltoside is commercially available in many countries under the tradename Monofer®, Monoferrio® or Diafer®.

The iron oligoisomaltoside complexes of the invention have been found to have properties which turn out to be advantageous when it comes to their medical use. In particular, total amount of free iron was found to be less than 0.01% w/v and in particular preferably less than 0.003% w/v for a 100 mg/mL solution of the iron oligoisomaltoside complex.

Furthermore, the strength of the iron oligoisomaltoside complexes of the invention was observed to be sufficiently high for them to release the iron under physiological conditions in vivo in an expedient manner once they are administered to human or non-human subjects. There are in vitro tests which allow to assess the strength in an accelerated manner. In one test, the complexes are subjected to hydrochloric acid hydrolysis under defined conditions (0.24 M HCl; 0.9% NaCl). The assay then determines the time elapsed until half of the iron carbohydrate complex in solution has dissociated into its component parts; iron and carbohydrate. This can be done by measuring the optical absorbance at 287.3 nm. The duration of time (T½), as measured in vitro, is a surrogate measure of the relative rate of dissociation of the iron carbohydrate complex after administration in vivo, i.e., it is a measure of the complex strength. In this test, the iron oligoisomaltoside complexes of the invention were found to have half-lives (T½) of at least 20, preferably at least 25, more preferably at least 30 hours. Expediently, a complex suitable for use in the present invention has such half-lives. This ensures reducing free iron toxicity while the iron from the iron complex compound is absorbed. On the other hand, half-lives (T½) of no more than 60, preferably of no more than 50, more preferably of no more than 40 hours, also provide significant advantages when it comes to enabling an expedient uptake of the iron into the body. Half-lives in the range of 25-35 hours are particularly preferred.

Another particular iron carbohydrate complex for use in this invention is ferric bepectate (FBP). Ferric bepectate is disclosed, for instance, in WO 2012/175608 A1, all of which is incorporated by reference. Iron complexes with dextran glucoheptonic acid represent further particular iron carbohydrate complex for use in this invention. These are also known as gleptoferron, which is a commercially available iron carbohydrate complex for se in pigs. Ferric dextran glucoheptonic acid such as gleptoferron is disclosed, for instance, in U.S. Pat. No. 3,639,588.

According to another group of embodiments, the iron complex compound is a polymeric ligand-substituted oxo-hydroxy iron complex compound. Polymeric ligand-substituted oxo-hydroxy iron complex compounds comprise or basically consist of iron ions (e.g., Fe³⁺), ligands and oxo and/or hydroxyl groups. The iron ions, oxo and/or hydroxyl groups form poly oxo-hydroxy iron particles. The ligands are incorporated therein through substitution of part of the initially present oxo or hydroxyl groups. This substitution is generally non-stoichiometric, occurs through formal bonding and leads to distinct alterations in the chemistry, crystallinity and material properties of the oxo-hydroxy iron. Polymeric ligand-substituted oxo-hydroxy iron complex compounds are described, for example, in WO 2008/096130 A1.

The average molar ratio of ligand to iron is typically in the range of from 10:1 to 1:10, such as in the range of from 5:1 to 1:5, from 4.1 to 1:4, from 3.1 to 1:3, from 2:1 to 1:2 or at about 1:1.

### Preparation of Iron Complex Compounds

Iron complex compounds of the invention can be prepared by contacting an iron preparation with a ligand in the presence of water. Iron preparations comprising iron in the form of an iron hydroxide and/or iron oxide-hydroxide can be used directly for this step. For example, a precipitate of iron hydroxide (e.g., ferric hydroxide) and/or iron oxide-hydroxide in an aqueous solution is contacted with a ligand (e.g., a carbohydrate preparation), followed by heating and raising the pH so as to form an iron complex compound (e.g., an iron complex compound comprising FeO(OH) cores). Alternatively, the iron hydroxide and/or iron oxide-hydroxide of the iron preparation is converted into a water-soluble iron salt as described herein by contacting the iron preparation with an acid. Expediently this conversion is performed in an aqueous solution comprising the reactants (iron hydroxide and/or iron oxide-hydroxide and acid). The choice of the acid depends on the iron salt to be produced. For example, iron chloride can be prepared by reacting the iron hydroxide and/or iron oxide-hydroxide of an iron preparation with hydrochloric acid. The reagents which, in addition to the iron preparation, are used in step (ii) of the process of the invention for preparing the iron complex compound are expediently substantially free of non-iron impurities such as arsenic, chromium, lead, mercury, cadmium and/or aluminium.

Iron carbohydrate complex compounds of the invention can thus be prepared by
(1) providing an aqueous solution that comprises a carbohydrate and an iron preparation as described herein comprising a water-soluble iron salt (e.g. ferric chloride),
(2) adding a base to the aqueous solution so as to form iron hydroxide, and
(3) then heating the aqueous solution so as to form the iron carbohydrate complex compound.

Preferably, the pH of the aqueous solution in step (1) is acidic, e.g., the solution is at a pH of 2 or lower, so as to prevent the precipitation of iron hydroxides. The addition of a base in step (2) is preferably performed in a slow or gradual manner so as to increase the pH to, for example, a pH of 5 or more, such as up to pH 11, 12, 13 or 14. Such gradual increase can be achieved by first adding a weak base (e.g., an alkali metal carbonate or alkaline earth metal carbonate such as sodium carbonate, potassium carbonate, sodium bicarbonate or potassium bicarbonate, or ammonium carbonate or ammonium bicarbonate, or ammonia) to increase the pH, e.g., up to pH 2-4, e.g., up to 2-3, and then further increasing the pH by adding a strong base (e.g., an alkali metal hydroxide or alkaline earth metal hydroxide such as sodium hydroxide, potassium hydroxide, calcium hydroxide or magnesium hydroxide).

Alternatively, iron carbohydrate complex compounds of the invention can be prepared by
(1) providing an aqueous solution that comprises a carbohydrate and an iron preparation as described herein comprising an iron hydroxide, iron oxide-hydroxide or a mixture thereof, and
(2) then heating the aqueous solution so as to form the iron carbohydrate complex compound.

The heating of the aqueous solution in the last step of the two above-described processes for preparing iron carbohydrate complex compounds of the invention facilitates the formation of the iron carbohydrate complex compound. For example, the aqueous solution may be heated to a temperature in the range of from 15° C. to boiling. Preferably, the temperature is gradually increased, for example in a first step the aqueous solution is heated to a temperature of from 15 to 70° C. and then is gradually heated further until boiling. To finalize the reaction the pH can be reduced to, e.g., pH 5-7 by adding an acid such as, for example, HCl or aqueous hydrochloric acid. In one embodiment, said reduction of pH is performed when the solution has been heated to about 50° C. and before it is further heated.

After heating, the product can be further processed by filtration and its pH can be adjusted to a neutral or sightly acidic pH (e.g., pH 5 to 7) by adding a base or acid such as those mentioned above. Further optional steps include purification, in particular the removal of salts, which may be achieved by ultrafiltration or dialysis, and sterilization which may be achieved by sterile filtration and/or heat treatment (e.g., at temperatures of 121° C. or higher). The purified solution can be used directly for preparing pharmaceutical compositions. Alternatively, solid iron carbohydrate complex can be obtained by precipitation, e.g., by adding an alcohol such as ethanol, or by drying, e.g. spray-drying.

The iron carbohydrate complex compound can be stabilised by mixing it with an organic hydroxyl acid or salt thereof such as citric acid, a citrate gluconic acid or a gluconate.

Accordingly, a typical process for producing the iron oligoisomaltos(id)es comprises the following steps:
(a) hydrolysis of dextran to obtain a hydrolysate;
(b) fractionation of the hydrolysate to obtain the oligoisomaltose; and
(c) hydrogenation of the oligoisomaltose to obtain the oligoisomaltoside
(d) iron complex formation, with the hydrogenation step being included if the process is for producing an iron oligoisomaltoside.

Further optional steps are:
Purification, e.g., diafiltration, to reduce the level of mono- and disaccharide in the oligoisomaltoside.
Purification, e.g., by ion exchange, to obtain a purified oligoisomaltoside.
Heating of the complex.
Filtration of the heated complex.
Membrane filtration to obtain a purified complex.
Addition of an organic acid such as citrate to obtain a stabilised complex.
Spray drying to obtain the complex as a solid, e.g., a powder.

For instance, iron oligoisomaltos(id)es such as the iron octasaccharide of the invention can be manufactured by contacting the disclosed oligoisomaltos(id)es with ferric chloride in water. Na₂CO₃ is then added followed by NaOH to reach a pH of about 10.5. Heating gives a black or dark brown colloidal solution, which can then be neutralized using HCl and filtered. Residues of unbound octasaccharide, free iron, and inorganic salts can be removed by membrane filtration. Citric acid monohydrate may be added to further stabilise the complex. Adjustment to a neutral or slightly acidic pH will result in a solution that can then be converted into solid form, e.g., a powder. To this end, the solution can be spray dried to give a black to dark brown powder.

Iron oligoisomaltoside are obtainable as described, for instance, in WO 2010/108493 A1 and WO 2019/048674 A1, all of which is incorporated by reference.

Ferric bepectate and its manufacture are disclosed, for instance, in WO 2012/175608 A1, all of which is incorporated by reference. Briefly, hydroxyethyl starch is dissolved in water. Then, the pH value is adjusted to a value of 8.0 to 10.0. Afterwards, a cyanide compound is added to the hydroxyethyl starch solution. Then the solution is heated to a temperature of 80 to 99° C. and kept at this temperature for a first time period. Finally, the pH value is adjusted to a value of 2.0 to 4.0 and the solution is brought to a temperature of 50 to 90° C. and kept at this temperature for a second time period. A process for manufacturing this heptonic-acid modified hydroxyethyl starch, HES 70/0.5, is described in Example 1, and the formation of the iron complex in Example 2, of WO 2012/175608 A1, all of which is incorporated by reference.

Polymeric ligand-substituted oxo-hydroxy iron complex compounds of the invention can be prepared by contacting an iron preparation as disclosed herein with a ligand in an aqueous solution at a first pH(A) and then changing the pH(A) to a second pH(B) to cause a solid precipitation of the polymeric ligand-substituted oxo-hydroxy iron complex compound. The solid precipitate can have a particulate, colloidal or sub-colloidal (nanoparticulate) structure.

The pH(A) is different from the pH(B). Preferably, the pH(A) is more acidic than the pH(B). For example, pH(A) is equal or below pH 2 and pH(B) is above pH 2. Starting from the pH at which oxo-hydroxy polymerization commences, the pH is preferably further increased to complete the reaction and promote precipitation of the polymeric ligand-substituted oxo-hydroxy iron complex compound formed. During said pH change further ligands and/or excipients can be added. Said pH change is preferably done in a gradual or stepwise manner, for example over a period of about 24 hours or over a period of about 1 hour, and in particular over a period of 20 minutes. The pH change can be effected by the addition of acids or bases. For example, the pH can be increased by adding sodium hydroxide, potassium hydroxide or sodium bicarbonate.

The polymeric ligand-substituted oxo-hydroxy iron complex compounds are typically produced in aqueous solutions, wherein the concentrations of iron ions and ligand are 1 μM or higher and in particular 1 mM or higher. The ratio of iron ions and ligand is chosen such that the relative amount of iron ions is not too high such that the rate of oxo-hydroxy polymerization occurs too rapidly, and efficient ligand incorporation is prevented, and the relative amount of ligand is not too high to prevent iron oxo-hydroxy polymerization. For example, the iron concentration is in the range of from 1 mM to 300 mM, such as from 20 mM to 200 mM and in particular at about 40 mM.

The ligands used for the formation of the polymeric ligand-substituted oxo-hydroxy iron complex compounds may have some buffering capacity which helps to stabilizing the pH range during complex formation. Buffering can also be achieved by adding an inorganic or organic buffering agent, which will not be involved in formal bonding with the iron ions, to the aqueous solution containing the iron preparation and the ligand. Typically, the concentration of such buffer, if present, is less than 500 mM or less than 200 mM, and in particular less than 100 mM.

The formation of the polymeric ligand-substituted oxo-hydroxy iron complex compounds typically takes place at a temperature within the range of from 20° C. to 120° C., e.g., 20° C. to 100° C., in particular from 20° C. to 30° C.

Optionally, the ionic strength in the aqueous solution comprising the iron preparation and the ligand can be increased by adding further electrolyte such as, e.g., potassium chloride or sodium chloride in an amount of, e.g., up to 10 wt-%, such as up to 2 wt-%, and in particular up to 1 wt-%.

The solid precipitate of polymeric ligand-substituted oxo-hydroxy iron complex compound can be separated and optionally be dried and processed further by, e.g., grinding before further use or formulation.

### Pharmaceutical Compositions

The invention further relates to a pharmaceutical composition comprising the iron complex compound of the invention and a pharmaceutically acceptable carrier.

Preferred are pharmaceutical compositions for parenteral use. These can be a ready-for-use fluid (fluid for injection or infusion); a fluid for dilution prior to use; or a solid for reconstitution. Ideally, such fluids are isotonic, sterile, pyrogen free and maintain suitable physical and chemical stability over the intended shelf-life. However, it is not always possible to meet all of these objectives and often it is required to balance opposite effects to find a “sweet spot” for a pharmaceutical composition to be suitable for its intended purpose.

Particularly preferred are ready-to-use injectable compositions.

Accordingly, pharmaceutical compositions of the invention include a fluid composition suitable for injection or infusion comprising the iron complex compound, water-for-injection and optionally further expedient excipients. Fluids include liquids, which are preferably presented as solutions (i.e., fluids and especially liquids wherein the iron complex compound is dissolved), These fluids can have the concentration of the iron complex compound that is desired to be administered. Alternatively, the concentration of the iron complex compound can be higher; such concentrate will require dilution with a suitable fluid prior to administration. The pharmaceutical compositions of the invention also include solids, such as powders, for reconstitution with a suitable fluid prior to administration. For instance, the pharmaceutical composition can be stored in spray-dried or lyophilised form and may then be reconstituted, typically as an aqueous composition, preferable a solution, suitable for parenteral administration, prior to administration to the subject. Such a composition can be reconstituted with sterile Water for Injection (NFI). Bacteriostatic reagents, such benzyl alcohol or phenol, may be included.

According to a preferred embodiment of the invention, the pharmaceutical composition is suitable for subcutaneous administration. Accordingly, particularly preferred are pharmaceutical ready-to-use injectable compositions for subcutaneous use. Usually, subcutaneous administration is limited by the total volume of fluid that is injected. If the amount of iron carbohydrate compound that is to be administered to a subject is relatively high (e.g., 10-30 mg iron/kg body weight in the form of an iron complex compound), this may require formulating a fluid with a relatively high concentration of iron complex compound in order to arrive at an acceptable injection volume. Generally, a given volume of fluid should contain as much iron as possible, to allow for the lowest possible injection volume. But relatively high concentrations of iron complex compound can result in viscous fluids that are difficult and/or painful to inject. Also, pH and osmolality considerations may require a lower iron concentration. Moreover, relatively high concentrations of iron complex compound can decrease the physical stability of the iron complex compound and thus result in a reduced shelf-life.

Expediently, the pharmaceutical compositions of the invention comprise 1 to 25%, preferably 2.5 to 20%; most preferably 2.5 to 7.5%, or 7.5 to 12.5%, or 15% to 20%; e.g., about 5% or about 10% or about 20%, (w/v) of elemental iron. Put differently, the concentration of the iron complex compound in the fluid pharmaceutical composition is 25 to 300 mg/mL, preferably 50 to 200 mg/mL, most preferably 75 to 150 mg/mL, e.g., about 100 mg/mL of elemental iron.

With a view on parenteral and especially subcutaneous administration, the pH of the fluid pharmaceutical composition is expediently in the range of 5.8 to 7.0, preferably 5.9 to 6.8; most preferably 5.9 to 6.6, e.g., at 6.0 to 6.4. Generally, it is preferred to choose the pH at the higher end of these ranges, i.e., towards a neutral pH. Therefore, injectable and infusible compositions should be suitably buffered if necessary. In this connection, sterile aqueous media that can be employed will be known to those of skill in the art in light of the present disclosure. For example, one dosage may be prepared with a volume of isotonic NaCl solution and sterile water prior to injection. For subcutaneous injection, the composition is typically administered without prior dilution (unless the size of the animal calls for a dose that would result in an injection volume that is too low to be administered). Typical subcutaneous injection volumes are 0.5 to 5 mL.

The turbidity of the fluid pharmaceutical composition is expediently below 2.0 NTU, preferably below 1.5 NTU, most preferably below 1.0 NTU, e.g., below 0.5 NTU.

Flow properties such as syringeability and injectability are characteristics to evaluate and control. Syringeability describes the ability of the composition to pass easily through a hypodermic needle on transfer from the vial prior to injection. It includes characteristics such as the ease of withdraw, clogging and foaming tendencies and accuracy of dose measurements. Increase in the viscosity and density, decreases the syringeability of the composition.

Injectability refers to the performance of the composition during injection and includes factors such as pressure or force required for injection. Evenness of flow, aspiration qualities, and freedom from clogging. The syringeability and injectability of the composition are closely related to the viscosity of the composition. A simple ejection of the composition into the open, if done very slowly with intermittent application of pressure to the plunger can provide certain information about the composition. Most methods used for injectability are qualitative in nature. A force monitoring device such as an Instron can be used to determine ejection and injection pressure, and the test results can be recorded on a X-Y recorder. Another instrument to assess the injectability measures the time required to smoothly inject a solution or suspension into meat under specified pressure from a syringe through a needle. When a test solution is injected through glass and plastic syringes of various sizes, regression equations are obtained of a given syringe type and diameter using needles of various gauge. These equations permit the calculation of the expected injection time for a given syringe needle system and for a given vehicle of a certain viscosity.

In order to provide compositions having a suitable syringeability, the fluid pharmaceutical compositions of the invention expediently have a viscosity of no greater than 60 cP. In another embodiment, the compositions have a viscosity of no greater than 50 cP, or no greater than 40 cP, or no greater than 30 cP, or no greater than 20 cP, or no greater than 40 cP, or no greater than 15 cP. In some embodiments, the compositions have a viscosity of between 1 cP and 50 cP, between 1 cP and 40 cP, between 1 cP and 30 cP, between 1 cP and 20 cP, between 1 cP and 15 cP, or between 1 cP and 10 cP at 25° C. In some embodiments, the compositions have a viscosity of about 50 cP, about 45 cP, about 40 cP, about 35 cP, about 30 cP, about 25 cP, about 20 cP, about 15 cP, or about 10 cP, or about 5 cP. In some embodiments, the compositions have a viscosity of between 10 cP and 50 cP, between 10 cP and 30 cP, between 10 cP and 20 cP, or between 5 cP and 15 cP.

“Viscosity” as used herein may be “kinematic viscosity” or “absolute viscosity.” “Kinematic viscosity” is a measure of the resistive flow of a fluid under the influence of gravity. When two fluids of equal volume are placed in identical capillary viscometers and allowed to flow by gravity, a viscous fluid takes longer than a less viscous fluid to flow through the capillary. If one fluid takes 200 seconds to complete its flow and another fluid takes 400 seconds, the second fluid is twice as viscous as the first on a kinematic viscosity scale. “Absolute viscosity”, sometimes called dynamic or simple viscosity, is the product of kinematic viscosity and fluid density: Absolute Viscosity=Kinematic Viscosity x Density. The dimension of kinematic viscosity is L^{2/T} where L is a length and T is a time. Commonly, kinematic viscosity is expressed in centistokes (cSt). The SI unit of kinematic viscosity is mm^{2/s}, which is 1 cSt. Absolute viscosity is expressed in units of centipoise (cP). The SI unit of absolute viscosity is the milliPascal-second (mPa-s), where 1 cP=1 mPa-s.

For reasons of storage, the fluid pharmaceutical compositions have a shelf-life of at least 1 year at 25° C., preferably at least 2 years at 25° C., more preferably at least 3 years at 25° C.

### Combination Therapy

Further described herein are combinations of an iron complex compound with one or more additional drugs. According to particular embodiments, the additional drug is selected from the group consisting of:
(1) erythropoiesis-stimulating agents (ESA), such as Erythropoietin (Epo), Epoetin alfa (Procrit/Epogen), Epoetin beta (NeoRecormon), Darbepoetin alfa (Aranesp), Methoxy polyethylene glycol-epoetin beta (Mircera), or those disclosed in US20210032305A, all of which is incorporated by reference;
(2) hepcidin modulators such as a hepcidin agonist or a hepcidin antagonist
(3) antiparasitic drugs, such as parasiticides, ectoparasiticide(s), and endoparasiticide(s);
(4) chemotherapeutic drugs;
(5) antibiotics;
(6) antivirals; and
(7) vaccines.

Those which are suitable for use in therapy of iron deficiency according to the invention represent a particular embodiment of this aspect of the invention. In cats with CKD, for instance, erythropoiesis-stimulating agents are indicated, and these would thus be administered in addition to an iron complex compound according to the invention if therapy of iron deficiency and especially of iron deficiency anaemia is required.

### Administration Regimens

The methods of therapy of iron deficiency in a subject according to the invention comprise administering a therapeutically effective amount of an iron complex compound to the subject in need of such therapy. Therefore, the methods of the invention may and, according to a preferred embodiment, do comprise, prior to said administration of the iron complex compound, determining whether said subject is iron-deficient, and administering said iron complex compound if said subject is iron-deficient.

Some variation in dosage will necessarily occur depending on the weight of the animal being treated. The person responsible for administration will, in any event, determine the appropriate dose. A typical treatment regimen of an iron complex compound would consist of a dose of 5 to 100 mg, such as 10 to 60 mg, in particular 15 to 25 mg, e.g., about 20 mg, of elemental iron per kg body weight. Alternatively, an effective amount of an iron complex compound is an amount of up to 50 mg iron/kg body weight, in particular up to 30 mg iron/kg body weight, or preferably up to 20 mg iron/kg body weight. Accordingly, a typical dose of an iron complex compound may be 10 to 2,800 mg elemental iron for a subject, such as a dog, weighing in the range of 0.5 to 140 kg.

The cumulative iron need can be determined using the Ganzoni formula and according to one embodiment, the calculated dose will be administered. Therefore, in some embodiments, an effective therapeutic amount of an iron complex compound is equal to a cumulative iron need. Such cumulative iron need may be lower or higher than a typical dose.

It is generally preferred that the dose is administered in a single setting (visit). Such (single) dose may be provided as a single (1) administration (e.g., injection), or alternatively in 2, 3 or more administrations (e.g., injections), depending on the dose volume. Generally, if the dose volume is greater than 5 mL, 7.5 mL or 10 mL, then the dose will preferably be split into 2, 3 or more administrations to lower the volume administered into each administration site. This is especially so for subcutaneous administration. The importance of splitting the dose and lowering the volume to be administered is variable, depending on the size of the subject, and the looseness of the skin of the subject. The person skilled in the art would know how to decide on the volume to be administered.

In another particular embodiment, the therapeutic methods of the invention comprise administering 2, or 3, or 4, or 5, or more doses over a period of time to ensure effective therapy of the ID or IDA, e.g., in a case where a single dose is insufficient or in a situation where the clinical signs of ID or IDA re-emerge after having previously disappeared and/or where ID or IDA is diagnosed anew in the same subject.

In a further particular embodiment, the therapeutic methods of the invention comprise administering several, repeated doses overtime to manage ID or IDA in a subject with a chronic blood loss caused by an underlying condition, e.g., a subject with CKD or a subject with IBD. Such a subject would potentially need iron continually (as maintenance therapy), and thus the therapy would need to be repeated regularly, on an ongoing basis.

For repeated dosing, a first dose of up to 50 mg iron/kg body weight, in particular up to 30 mg iron/kg body weight, or preferably up to 20 mg iron/kg body weight is followed by a second dose of up to 50 mg iron/kg body weight, in particular up to 30 mg iron/kg body weight, or preferably up to 20 mg iron/kg body weight. The two consecutive doses may be administered within 1 month, 2 weeks, or preferably 1 week.

Preferably, they are administered within one week. Further doses of up to 50 mg iron/kg body weight, in particular up to 30 mg iron/kg body weight, or preferably up to 20 mg iron/kg body weight. This further, e.g., third, dose(s) may be administered within the same time frame, i.e., 1 month, 2 weeks, or preferably 1 week. These multiple doses are preferably administered at least 2 and in particular 3 days apart. For instance, if 3 doses are to be administered within one week, it is preferred to administer these doses on day 1, 4 and 7.

According to the invention, the iron complex compounds can be administered parenterally, for instance, by intramuscular injection, intravenous (IV) bolus injection, or IV infusion. However, according to a preferred embodiment of the invention, the parenteral administration of the iron complex compound is subcutaneous administration. For instance, a convenient site for subcutaneous administration is governed by the relatively loose skin as, for instance, in the area laterally above the dorsal plane behind the shoulder blades over the ribs of companion animals such as dogs. Alternatively, the dorsal paralumbar region could be used for injection. Other typical areas for subcutaneous injection are known by those skilled in the art.

Administration “in combination with” one or more further therapeutic agents includes simultaneous (concurrent) and consecutive or sequential administration in any order. The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes. The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes. As used herein, “in conjunction with” refers to administration of one treatment modality in addition to another treatment modality. As such, “in conjunction with” refers to administration of one treatment modality before, during or after administration of the other treatment modality to the animal.

### Ferric Octasaccharide

In certain aspects, the invention specifically relates to ferric octasaccharide complexes and pharmaceutical compositions comprising a ferric octasaccharide complex. The ferric octasaccharide complexes are particularly useful in the methods of therapy described herein. All of the below references to the ferric octasaccharide of the invention apply equally to the use of the ferric octasaccharide in the methods of therapy disclosed herein.

The ferric octasaccharide comprises iron complexed with an octasaccharide. Preferably, the ferric octasaccharide comprises an iron oxide hydroxide in stable association with an octasaccharide. In some embodiments, the ferric octasaccharide comprises stabilizers such as organic acids. Preferably, the organic acid is an organic hydroxy acid. Suitable examples of organic hydroxy acids are gluconic acid and citric acid. Citric acid is an expedient example. If present, the amount of citric acid is typically in the range of 3 to 20% by weight of total quantity of elemental iron.

In some embodiments, the ferric octasaccharide comprises a salt such as a metal chloride. The metal chloride may be sodium chloride or potassium chloride. Preferably, the metal chloride is sodium chloride. If present, the amount of sodium chloride is typically in the range of 3 to 110% by weight of the total quantity of elemental iron.

In some embodiments, the ferric octasaccharide comprises water. If present, the amount of water is typically in the range of 3 to 25% by weight of the total quantity of elemental iron.

In particular embodiments, the ferric octasaccharide comprises stabilizers, salt/s and water. In a preferred embodiment, the ferric octasaccharide comprises citric acid, sodium chloride and water.

In one aspect of the invention, there is provided a ferric octasaccharide having the formula:
{FeOOH, (Octasaccharide)_{Q}}, that optionally contains a stabilizer and/or a metal chloride and/or H₂O, wherein
Q is 0.06 to 0.11, in particular 0.07 to 0.10, preferably 0.08 to 0.09, more preferably about 0.085.

In a preferred embodiment, the ferric octasaccharide of the invention has the formula:
{FeOOH, (Octasaccharide)_{Q}, (C₆H₈O₇)_{R}}, that optionally contains a metal chloride and/or H₂O, wherein
Q is 0.06 to 0.11, in particular 0.07 to 0.10, preferably 0.08 to 0.09, more preferably about 0.085; and
R is 0.02 to 0.04, in particular 0.025 to 0.038, preferably 0.028 to 0.034, more preferably about 0.031.

In another preferred embodiment, the ferric octasaccharide of the invention has the formula:
{FeOOH, (Octasaccharide)_{Q}, (C₆H₈O₇)_{R}}, that contains a metal chloride and H₂O, wherein
Q is 0.06 to 0.11, in particular 0.07 to 0.10, preferably 0.08 to 0.09, more preferably about 0.085;
R is 0.02 to 0.04, in particular 0.025 to 0.038, preferably 0.028 to 0.034, more preferably about 0.031; and
the metal chloride is sodium chloride or potassium chloride, preferably sodium chloride.

In another preferred embodiment, the ferric octasaccharide of the invention has the formula:
{FeOOH, (Octasaccharide)_{Q}, (C₆H₈O₇)_{R}}, (H₂O)_{X}, (MeCl)_{Y}, wherein
Q is 0.06 to 0.11, in particular 0.07 to 0.10, preferably 0.08 to 0.09, more preferably about 0.085;
R is 0.02 to 0.04, in particular 0.025 to 0.038, preferably 0.028 to 0.034, more preferably about 0.031;
X is 0.15 to 0.55, in particular 0.25 to 0.45, preferably 0.30 to 0.40, more preferably about 0.34;
Y is 0.05 to 1, in particular 0.05 to 0.50, preferably 0.09 to 0.40, preferably 0.09 to 0.30, more preferably 0.09 to 0.20, even more preferably about 0.14; and
Me is a monovalent metal ion such as a sodium ion or potassium ion and is preferably a sodium ion.

In another preferred embodiment, the ferric octasaccharide of the invention has the formula:
{FeOOH, (Octasaccharide)_{Q}, (C₆H₈O₇)_{R}}, (H₂O)_{X}, (MeCl)_{Y}, wherein
Q is 0.08 to 0.09, preferably about 0.085;
R is 0.028 to 0.034, preferably about 0.031;
X is 0.30 to 0.40, preferably about 0.34; and
Y is 0.05 to 1, in particular 0.05 to 0.50, preferably 0.09 to 0.40, preferably 0.09 to 0.30, more preferably 0.09 to 0.20, even more preferably about 0.14; and
Me is a sodium ion.

In another preferred embodiment, the ferric octasaccharide of the invention has the formula:
{FeOOH, (Octasaccharide)_{Q}, (C₆H₈O₇)_{R}}, (H₂O)_{X}, (NaCl)_{Y}, wherein
Q is about 0.085;
R is about 0.031;
X is about 0.34; and
Y is about 0.14.

In a particularly preferred embodiment, the ferric octasaccharide of the invention has the formula:
{FeOOH, (C₆H₁₀O₆)_{T}—(C₆H₁₀O₅)_{Z}—(C₆H₁₃O₅)_{T}, (C₆H₈O₇)_{R}}, (H₂O)_{X}, (MeCl)_{Y}, wherein
T is 0.06 to 0.11, in particular 0.07 to 0.10, preferably 0.08 to 0.09, more preferably about 0.085;
Z is 0.25 to 0.75, in particular 0.35 to 0.65, preferably 0.45 to 0.55, even more preferably about 0.51;
R is 0.02 to 0.04, in particular 0.025 to 0.038, preferably 0.028 to 0.034, more preferably about 0.031;
X is 0.15 to 0.55, in particular 0.25 to 0.45, preferably 0.30 to 0.40, more preferably about 0.34;
Y is 0.05 to 1, in particular 0.05 to 0.50, preferably 0.09 to 0.40, preferably 0.09 to 0.30, more preferably 0.09 to 0.20, even more preferably about 0.14; and
Me is a monovalent metal ion such as a sodium ion or potassium ion, and is preferably a sodium ion.

In another particularly preferred embodiment, the ferric octasaccharide of the invention has the formula:
{FeOOH, (C₆H₁₀O₆)_{T}—(C₆H₁₀O₅)_{Z}—(C₆H₁₃O₅)_{T}, (C₆H₈O₇)_{R}}, (H₂O)_{X}, (NaCl)_{Y}, wherein
T is 0.08 to 0.09, preferably about 0.085;
Z is 0.45 to 0.55, preferably about 0.51;
R is 0.028 to 0.034, preferably about 0.031;
X is 0.30 to 0.40, preferably about 0.34; and
Y is 0.05 to 1, in particular 0.05 to 0.50, preferably 0.09 to 0.40, preferably 0.09 to 0.30, more preferably 0.09 to 0.20, even more preferably about 0.14.

In another particularly preferred embodiment, the ferric octasaccharide of the invention has the formula:
{FeOOH, (C₆H₁₀O₆)_{T}—(C₆H₁₀O₅)_{Z}—(C₆H₁₃O₅)_{T}, (C₆H₈O₇)_{R}}, (H₂O)_{X}, (NaCl)_{Y}, wherein
T is about 0.085;
Z is about 0.51;
R is about 0.031;
X is about 0.34; and
Y is about 0.14.

In particular embodiments, the ferric octasaccharide comprises a mixture of oligoisomaltosides having a weight average molecular weight in the range of 1,150 to 1,350 Da. In particular embodiments, the octasaccharide comprises a mixture of oligoisomaltosides having a weight average molecular weight in the range of 1,200 to 1,300 Da, preferably from 1,225 to 1275 Da; e.g., of about 1250 Da.

In some embodiments, the ferric octasaccharide has an “apparent” peak molecular weight (Mp measured by Gel Permeation Chromatography) in the range of 125,000 to 185,000 Da. In particular embodiments, the “apparent” peak molecular weight (Mp measured by Gel Permeation Chromatography) of the ferric octasaccharide is in the range 135,000 to 175,000 Da, preferably 140,000 to 155,000 Da. In certain embodiments, the “apparent” peak molecular weight (Mp) is in the range of 145,000 to 155,000 Da.

In some embodiments, the ferric octasaccharide has a monosaccharide and disaccharide content of less than 10.0% by weight of the octasaccharide. In particular embodiments, the content of monomer and dimer is less than 3.0%, preferably less than 1.0%; e.g., 0.1 to 0.5%, by weight of the octasaccharide.

In some embodiments, the fraction of ferric octasaccharide with more than 9 monosaccharide units is less than 40% by weight of the octasaccharide. In particular embodiments, the fraction with more than 9 monosaccharide units is less than 35%, preferably less than 30%; e.g., 20 to 30%, by weight of the octasaccharide.

In some embodiments of the ferric octasaccharide, at least 40% by weight of the oligoisomaltoside molecules have 6-10 monosaccharide units. In particular embodiments, the proportion of molecules having 6-10 monosaccharide units is at least 45%; e.g., from 40 to 60% or from 45 to 55%, by weight of the octasaccharide.

In some embodiments, the proportion of molecules having 6-10 monosaccharide units is higher by weight than the proportion of molecules having 3-6 monosaccharide units.

In some embodiments, the dispersity (Mw/Mn) of the ferric octasaccharide is in the range of 1.05 to 1.4. In particular embodiments, the dispersity (Mw/Mn) is in the range of 1.1 to 1.3; e.g., at about 1.2.

In some embodiments, the amount of reducing sugar in the ferric octasaccharide is 2.5% or less by weight of the octasaccharide. In particular embodiments, the amount of reducing sugar is 2.5% or less; preferably 1.0% or less; more preferably 0.5% or less; e.g. about 0.3%, by weight of the octasaccharide.

In some embodiments, the amount of reducing sugar in the ferric octasaccharide prior to hydrogenation is (i) at least 10% or at least 15% and (ii) less than 35%; preferably no more than 30%; e.g. 10% to 30% or preferably 15 to 25%, by weight of the octasaccharide.

In some embodiments, the ferric octasaccharide contains 10 to 50%; preferably 15 to 35%; most preferably 20 to 30%; e.g., 20 to 25%, of iron by weight of the ferric octasaccharide. In some embodiments, the weight ratio of elemental iron to octasaccharide in the ferric octasaccharide is 10:90 to 50:50; preferably 15:85 to 45:55; most preferably 20:80 to 40:60; e.g., about 70:30.

In some embodiments, the total amount of free iron in the ferric octasaccharide is 0.01% w/v or less; preferably less than 0.003% w/v, for a 100 mg/mL solution.

In another aspect of the invention, there is provided a ferric octasaccharide comprising iron complexed with an octasaccharide, wherein (i) the octasaccharide has a weight average molecular weight in the range of 1,150 to 1,350 Da; (ii) the content of monosaccharide and disaccharide is less than 10.0% by weight of the octasaccharide; (iii) the fraction with more than 9 monosaccharide units is less than 40% by weight of the octasaccharide; (iv) at least 40% by weight of the molecules have 6-10 monosaccharide units; (v) the “apparent” peak molecular weight (Mp) of the octasaccharide complex is in the range of 125,000 to 185,000 Da; (vi) the dispersity (Mw/Mn) of the complex is in the range of 1.05 to 1.4; and (vii) the amount of reducing sugar is 2.5% or less by weight of the octasaccharide.

All of the above embodiments, apply equally to this aspect.

### EXEMPLARY EMBODIMENTS

1. A method of therapy of iron deficiency in a companion animal, which method comprises administering an iron complex compound.

2. The method of embodiment 1, wherein the companion animal is a canine, feline or equine.

3. The method of embodiment 1, wherein the companion animal is a dog or a cat.

4. The method of embodiment 1, wherein the companion animal is a dog.

5. The method of any one of embodiments 1-4, wherein the companion animal has a reticulocyte haemoglobin content (CHr)/reticulocyte haemoglobin equivalent (RET-He) of 20 pg or less.

6. The method of any one of embodiments 1-4, wherein the iron deficiency is iron deficiency anaemia.

7. The method of any one of embodiments 1-6, wherein the companion animal, preferably a dog or a cat, has a haematocrit (HCT/PCV) of less than 35%.

8. The method of any one of embodiments 1-7, wherein the companion animal, preferably a dog or a cat, has a haemoglobin concentration (Hb) of less than 12 g/dL.

9. The method any one of embodiments 1-8, wherein the companion animal, preferably a dog or a cat, has a mean corpuscular volume (MCV) of less than 60 fL.

10. The method any one of embodiments 1-9, wherein the companion animal, preferably a dog or a cat, has a mean corpuscular haemoglobin concentration (MCHC) of 30 g/dL or less.

11. The method any one of embodiments 1-10, wherein the dose is 5 to 100 mg per kg body weight; preferably 10 to 60 mg per kg body weight; most preferably 15 to 25 mg per kg body weight; e.g., about 20 mg per kg body weight.

12. The method of any one of embodiments 1-11, wherein the dose is up to 50 mg iron/kg body weight; preferably up to 30 mg iron/kg body weight; most preferably up to 20 mg iron/kg body weight.

13. The method of any one of embodiments 1-12, wherein the dose is a single dose.

14. The method of any one of embodiments 1-13, wherein the dose is provided as a single (1) administration, preferably injection and especially subcutaneous injection.

15. The method of any one of embodiments 1-13, wherein the dose is provided as 2, 3, or more administrations, preferably injections and especially subcutaneous injections.

16. The method of any one of embodiments 1-12, wherein more than one dose is administered.

17. The method of embodiment 16, wherein the more than one dose is a dose of up to 50 mg iron/kg body weight; preferably up to 30 mg iron/kg body weight; most preferably up to 20 mg iron/kg body weight.

18. The method of embodiment 16 or 17, wherein two consecutive doses are administered within 1 month; preferably within 2 weeks; most preferably within 1 week.

19. The method of any one of embodiments 1-18, wherein the administration is subcutaneous administration.

20. The method of embodiment 19, wherein the site of subcutaneous administration is in the area laterally above the dorsal plane behind the shoulder blades over the ribs or in the dorsal paralumbar region.

21. The method of any one of embodiments 1-20, wherein the iron complex compound is an iron carbohydrate complex.

22. The method of embodiment 21, wherein the carbohydrate is an oligosaccharide.

23. The method of embodiment 22, wherein the oligosaccharide is an oligoisomaltose.

24. The method of embodiment 23, wherein the oligoisomaltose is a hydrogenated oligoisomaltose (oligoisomaltoside).

25. The method of any one of embodiments 22-24, wherein the oligosaccharide has a weight average molecular weight (Mw) in the range of 850 to 1,150 Da; preferably from 950 to 1,050 Da; most preferably from 975 to 1025 Da; e.g., of about 1000 Da.

26. The method of embodiment 25, wherein the proportion of molecules having 3-6 monosaccharide units is higher by weight than the proportion of molecules having 6-10 monosaccharide units.

27. The method of embodiment 25 or 26, wherein the proportion of molecules having 3-6 monosaccharide units is at least 40%; preferably at least 50%; e.g., from 40 to 70% or from 50 to 70%, by weight of the oligosaccharide.

28. The method of any one of embodiments 25 to 27, wherein the fraction with more than 9 monosaccharide units is less than 30%; preferably less than 25%; most preferably less than 20%; e.g., 5% to 15%, by weight of the oligosaccharide.

29. The method of any one of embodiments 22-24, wherein the oligosaccharide has a weight average molecular weight (Mw) in the range of 1,150 to 1,350 Da; preferably from 1,200 to 1,300 Da; most preferably from 1,225 to 1275 Da; e.g., of about 1250 Da.

30. The method of embodiment 29, wherein the proportion of molecules having 6-10 monosaccharide units is higher by weight than the proportion of molecules having 3-6 monosaccharide units.

31. The method of embodiment 29 or 30, wherein the proportion of molecules having 6-10 monosaccharide units is at least 40%; preferably at least 45%; e.g., from 40 to 60% or from 45 to 55%, by weight of the oligosaccharide.

32. The method of any one of embodiments 29-31, wherein the fraction with more than 9 monosaccharide units is less than 40%; preferably less than 35%; most preferably less than 30%; e.g., 20 to 30%, by weight of the oligosaccharide.

33. The method of any one of embodiments 22-32, wherein the content of monomer and dimer is less than 10.0%; preferably less than 3.0%; most preferably less than 1.0%; e.g., 0.1 to 0.5%, by weight of the oligosaccharide.

34. The method of any one of embodiments 22-33, wherein the amount of reducing sugar is 2.5% or less; preferably 1.0% or less; more preferably 0.5% or less; e.g. about 0.3%, by weight of the oligosaccharide.

35. The method of any one of embodiments 22-33, wherein the amount of reducing sugar is (i) at least 10% or at least 15% and (ii) less than 35%; preferably no more than 30%; e.g. 10% to 30% or preferably 15 to 25%, by weight of the oligosaccharide.

36. The method of any one of embodiments 22-35, wherein the iron oligosaccharide complex contains 10 to 50%; preferably 15 to 35; most preferably 20 to 30%; e.g., 20 to 25%, of iron by weight of the iron oligosaccharide complex.

37. The method of any one of embodiments 22-36, wherein the weight ratio of elemental iron to oligosaccharide in the iron oligosaccharide complex is 10:90 to 50:50; preferably 15:85 to 45:55; most preferably 20:80 to 40:60; e.g., about 70:30.

38. The method of any one of embodiments 22-37, wherein the “apparent” peak molecular weight (Mp measured by Gel Permeation Chromatography) of the iron oligosaccharide complex is in the range 120,000 to 190,000 Da; preferably 130,000 to 180,000 Da; or preferably 125,000 to 185,000 Da, more preferably 135,000 to 175,000 Da most preferably 140,000 to 155,000 Da.

39. The method of any one of embodiments 22-38, wherein the “apparent” peak molecular weight (Mp measured by Gel Permeation Chromatography) of the iron oligosaccharide complex is in the range 145,000 to 155,000 Da.

40. The method of any one of embodiments 22-39, wherein the dispersity (Mw/Mn) is in the range of 1.0 to 1.5; preferably 1.05 to 1.4; more preferably 1.1 to 1.3; e.g., at about 1.2.

41. The method of any one of embodiments 22-40, wherein the iron oligosaccharide complex contains citric acid.

42. The method of embodiment 41, wherein the amount of citric acid is 3 to 20% by weight of total quantity of elemental iron.

43. The method of any one of embodiments 22-42, wherein the total amount of free iron is 0.01% w/v or less; preferably less than 0.003% w/v, for a 100 mg/mL solution of the iron oligosaccharide complex.

44. The method of any one of embodiments 22-43, wherein the iron oligosaccharide complex contains sodium chloride.

45. The method of any one of embodiments 22-44, wherein the iron oligosaccharide complex contains water.

46. The method of any one of embodiments 22-24 or 29-45, wherein the iron oligosaccharide complex has the formula:
{FeOOH, (Octasaccharide)_{Q}}, that optionally contains a stabilizer and/or a metal chloride and/or H₂O, wherein
Q is 0.06 to 0.11, in particular 0.07 to 0.10, preferably 0.08 to 0.09, more preferably about 0.085.

47. The method of embodiment 46, wherein the iron oligosaccharide complex contains citric acid.

48. The method of any one of embodiments 22-24 or 29-47, wherein the iron oligosaccharide complex has the formula:
{FeOOH, (Octasaccharide)_{Q}, (C₆H₈O₇)_{R}}, that optionally contains a metal chloride and/or H₂O, wherein
Q is 0.06 to 0.11, in particular 0.07 to 0.10, preferably 0.08 to 0.09, more preferably about 0.085; and
R is 0.02 to 0.04, in particular 0.025 to 0.038, preferably 0.028 to 0.034, more preferably about 0.031.

49. The method of any one of embodiments 46 to 48, wherein the iron oligosaccharide complex contains sodium chloride.

50. The method of any one of embodiments 46 to 49, wherein the iron oligosaccharide complex contains H₂O.

51. The method of any one of embodiments 22-24 or 29-50, wherein the iron oligosaccharide complex has the formula:
{FeOOH, (Octasaccharide)_{Q}, (C₆H₈O₇)_{R}}, (H₂O)_{X}, (MeCl)_{Y}
Q is 0.06 to 0.11, in particular 0.07 to 0.10, preferably 0.08 to 0.09, more preferably about 0.085;
R is 0.02 to 0.04, in particular 0.025 to 0.038, preferably 0.028 to 0.034, more preferably about 0.031;
X is 0.15 to 0.55, in particular 0.25 to 0.45, preferably 0.30 to 0.40, more preferably about 0.34;
Y is 0.05 to 1, in particular 0.05 to 0.50, preferably 0.09 to 0.40, preferably 0.09 to 0.30, more preferably 0.09 to 0.20, even more preferably about 0.14; and
Me is a monovalent metal ion.

52. The method of embodiment 51, wherein the monovalent metal ion is a sodium ion or potassium ion.

53. The method of embodiment 52, wherein the monovalent metal ion is a sodium ion.

54. The method of any one of embodiments 51-53, wherein:
Q is 0.08 to 0.09, preferably about 0.085;
R is 0.028 to 0.034, preferably about 0.031;
X is 0.30 to 0.40, preferably about 0.34;
Y is 0.05 to 1, in particular 0.05 to 0.50, preferably 0.09 to 0.40, preferably 0.09 to 0.30, more preferably 0.09 to 0.20, even more preferably about 0.14; and
Me is a sodium ion.

55. The method of any one of embodiments 22-24 or 29-54, wherein the iron oligosaccharide complex has the formula:
{FeOOH, (C₆H₁₀O₆)_{T}—(C₆H₁₀O₅)_{Z}—(C₆H₁₃O₅)_{T}, (C₆H₈O₇)_{R}}, (H₂O)_{X}, (MeCl)_{Y}, wherein
T is 0.06 to 0.11, in particular 0.07 to 0.10, preferably 0.08 to 0.09, more preferably about 0.085;
Z is 0.25 to 0.75, in particular 0.35 to 0.65, preferably 0.45 to 0.55, even more preferably about 0.51;
R is 0.02 to 0.04, in particular 0.025 to 0.038, preferably 0.028 to 0.034, more preferably about 0.031;
X is 0.15 to 0.55, in particular 0.25 to 0.45, preferably 0.30 to 0.40, more preferably about 0.34;
Y is 0.05 to 1, in particular 0.05 to 0.50, preferably 0.09 to 0.40, preferably 0.09 to 0.30, more preferably 0.09 to 0.20, even more preferably about 0.14; and
Me is a monovalent metal ion such as a sodium ion or potassium ion, and is preferably a sodium ion.

56. The method of any one of embodiments 22-24 or 29-55 wherein the iron oligosaccharide complex has the formula:
{FeOOH, (C₆H₁₀O₆)_{T}—(C₆H₁₀O₅)_{Z}—(C₆H₁₃O₅)_{T}, (C₆H₈O₇)_{R}}, (H₂O)_{X}, (NaCl)_{Y}, wherein
T is 0.08 to 0.09, preferably about 0.085;
Z is 0.45 to 0.55, preferably about 0.51;
R is 0.028 to 0.034, preferably about 0.031;
X is 0.30 to 0.40, preferably about 0.34; and
Y is 0.05 to 1, in particular 0.05 to 0.50, preferably 0.09 to 0.40, preferably 0.09 to 0.30, more preferably 0.09 to 0.20, even more preferably about 0.14.

57. A pharmaceutical composition comprising the iron complex compound of any one of embodiments 21-56 and a pharmaceutically acceptable carrier.

58. The pharmaceutical composition of embodiment 57, which is a solid, preferably powder, for reconstitution.

59. The pharmaceutical composition of embodiment 57, which is a ready-to use fluid or a fluid for dilution prior to use.

60. The pharmaceutical composition of any one of embodiments 57-59, which is suitable for subcutaneous administration.

61. The pharmaceutical composition of any one of embodiments 57-40, which comprises 1 to 25%; preferably 2 to 15%; most preferably 2.5 to 7.5 or 7.5 to 12.5; e.g., about 5% or about 10%, (w/v) of elemental iron.

62. The pharmaceutical composition of any one of embodiments 57-61, wherein the concentration of the iron complex compound is 25 to 300 mg/mL; preferably 50 to 200 mg/mL; most preferably 75 to 150 mg/mL; e.g. about 100 mg/mL of elemental iron.

63. The pharmaceutical composition of any one of embodiments 57-62, wherein the pH is 5.8 to 7.0; preferably 5.9 to 6.8; most preferably 5.9 to 6.6; e.g., 6.0 to 6.4.

64. The pharmaceutical composition of any one of embodiments 57-63, wherein the turbidity is below 2.0 NTU; preferably below 1.5 NTU; most preferably below 1.0 NTU; e.g. below 0.5.

65. The pharmaceutical composition of any one of embodiments 57-64, which has a viscosity of no greater than 60 cP.

66. The pharmaceutical composition of any one of embodiments 57-65, which has a shelf-life of at least 3 years at 25° C.

67. Ferric octasaccharide comprising iron complexed with an octasaccharide, wherein (i) the octasaccharide has a weight average molecular weight in the range of 1,150 to 1,350 Da; (ii) the content of monosaccharide and disaccharide is less than 10.0% by weight of the octasaccharide; (iii) the fraction with more than 9 monosaccharide units is less than 40% by weight of the octasaccharide; (iv) at least 40% by weight of the molecules have 6-10 monosaccharide units; (v) the “apparent” peak molecular weight (Mp) of the octasaccharide complex is in the range of 125,000 to 185,000 Da; (vi) the dispersity (Mw/Mn) of the complex is in the range of 1.05 to 1.4; and (vii) the amount of reducing sugar is 2.5% or less by weight of the octasaccharide.

68. The ferric octasaccharide of embodiment 67, wherein the octasaccharide has a weight average molecular weight (Mw) in the range of 1,200 to 1,300 Da; more preferably from 1,225 to 1275 Da; e.g., of about 1250 Da.

69. The ferric octasaccharide of embodiment 67 or 68, wherein the proportion of molecules having 6-10 monosaccharide units is higher by weight than the proportion of molecules having 3-6 monosaccharide units.

70. The ferric octasaccharide of any one of embodiments 67-69, wherein the proportion of molecules having 6-10 monosaccharide units is at least 45%; e.g., from 40 to 60% or from 45 to 55%, by weight of the octasaccharide.

71. The ferric octasaccharide of any one of embodiments 67-70, wherein the fraction with more than 9 monosaccharide units is less than 35%; more preferably less than 30%; e.g., 20 to 30%, by weight of the octasaccharide.

72. The ferric octasaccharide of any one of embodiments 67-71, wherein the content of monomer and dimer is less than 3.0%; more preferably less than 1.0%; e.g., 0.1 to 0.5%, by weight of the octasaccharide.

73. The ferric octasaccharide of any one of embodiments 67-72, wherein the amount of reducing sugar is 2.5% or less; preferably 1.0% or less; more preferably 0.5% or less; e.g. about 0.3%, by weight of the octasaccharide.

74. The ferric octasaccharide of any one of embodiments 67-73, wherein the amount of reducing sugar prior to hydrogenation is (i) at least 10% or at least 15% and (ii) less than 35%; preferably no more than 30%; e.g. 10% to 30% or preferably 15 to 25%, by weight of the octasaccharide.

75. The ferric octasaccharide of any one of embodiments 67-74, wherein the ferric octasaccharide contains 10 to 50%; preferably 15 to 35; most preferably 20 to 30%; e.g., 20 to 25%, of iron by weight of the ferric octasaccharide.

76. The ferric octasaccharide of any one of embodiments 67-75, wherein the weight ratio of elemental iron to octasaccharide in the ferric octasaccharide is 10:90 to 50:50; preferably 15:85 to 45:55; most preferably 20:80 to 40:60; e.g., about 70:30.

77. The ferric octasaccharide of any one of embodiments 67-76, wherein the “apparent” peak molecular weight (Mp measured by Gel Permeation Chromatography) of the ferric octasaccharide is in the range 135,000 to 175,000 Da, more preferably 140,000 to 155,000 Da.

78. The method of any one of embodiments 67-77, wherein the “apparent” peak molecular weight (Mp measured by Gel Permeation Chromatography) of the iron oligosaccharide complex is in the range 145,000 to 155,000 Da.

79. The ferric octasaccharide of any one of embodiments 67-78, wherein the dispersity (Mw/Mn) is in the range of 1.1 to 1.3; e.g., at about 1.2.

80. The ferric octasaccharide of any one of embodiments 67-79, wherein the ferric octasaccharide contains citric acid.

81. The ferric octasaccharide of embodiment 80, wherein the amount of citric acid is 3 to 20% by weight of total quantity of elemental iron.

82. The ferric octasaccharide of any one of embodiments 67-81, wherein the total amount of free iron is 0.01% w/v or less; preferably less than 0.003% w/v, for a 100 mg/mL solution.

83. The ferric octasaccharide of any one of embodiments 67-82, wherein the ferric octasaccharide contains sodium chloride.

84. The ferric octasaccharide of any one of embodiments 67-83, wherein the ferric octasaccharide contains water.

85. The ferric octasaccharide of any one of embodiments 67-84, wherein the ferric octasaccharide has the formula:
{FeOOH, (Octasaccharide)_{Q}}, that optionally contains a stabilizer and/or a metal chloride and/or H₂O, wherein
Q is 0.06 to 0.11, in particular 0.07 to 0.10, preferably 0.08 to 0.09, more preferably about 0.085.

86. The ferric octasaccharide of embodiment 85, wherein the ferric octasaccharide contains citric acid.

87. The ferric octasaccharide of any one of embodiments 67-86, wherein the ferric octasaccharide has the formula:
{FeOOH, (Octasaccharide)_{Q}, (C₆H₈O₇)_{R}}, that optionally contains a metal chloride and/or H₂O, wherein
Q is 0.06 to 0.11, in particular 0.07 to 0.10, preferably 0.08 to 0.09, more preferably about 0.085; and
R is 0.02 to 0.04, in particular 0.025 to 0.038, preferably 0.028 to 0.034, more preferably about 0.031.

88. The ferric octasaccharide of any one of embodiments 85-87, wherein the ferric octasaccharide contains sodium chloride.

89. The ferric octasaccharide of any one of embodiments 85-88, wherein the ferric octasaccharide contains H₂O.

90. The ferric octasaccharide of any one of embodiments 67-89, wherein the ferric octasaccharide has the formula:
(FeOOH, (Octasaccharide)_{Q}, (C₆H₈O₇)_{R}), (H₂O)_{X}, (MeCl)_{Y}
Q is 0.06 to 0.11, in particular 0.07 to 0.10, preferably 0.08 to 0.09, more preferably about 0.085;
R is 0.02 to 0.04, in particular 0.025 to 0.038, preferably 0.028 to 0.034, more preferably about 0.031;
X is 0.15 to 0.55, in particular 0.25 to 0.45, preferably 0.30 to 0.40, more preferably about 0.34;
Y is 0.05 to 1, in particular 0.05 to 0.50, preferably 0.09 to 0.40, preferably 0.09 to 0.30, more preferably 0.09 to 0.20, even more preferably about 0.14; and
Me is a monovalent metal ion.

91. The ferric octasaccharide of embodiment 90, wherein the monovalent metal ion is a sodium ion or potassium ion.

92. The ferric octasaccharide of embodiment 91, wherein the monovalent metal ion is a sodium ion.

93. The ferric octasaccharide of any one of embodiments 90-92, wherein:
Q is 0.08 to 0.09, preferably about 0.085;
R is 0.028 to 0.034, preferably about 0.031;
X is 0.30 to 0.40, preferably about 0.34;
Y is 0.05 to 1, in particular 0.05 to 0.50, preferably 0.09 to 0.40, preferably 0.09 to 0.30, more preferably 0.09 to 0.20, even more preferably about 0.14; and
Me is a sodium ion.

94. The ferric octasaccharide of any one of embodiments 67-93, wherein the ferric octasaccharide has the formula:
{FeOOH, (C₆H₁₀O₆)_{T}—(C₆H₁₀O₅)_{Z}—(C₆H₁₃O₅)_{T}, (C₆H₈O₇)_{R}}, (H₂O)_{X}, (MeCl)_{Y}, wherein
T is 0.06 to 0.11, in particular 0.07 to 0.10, preferably 0.08 to 0.09, more preferably about 0.085;
Z is 0.25 to 0.75, in particular 0.35 to 0.65, preferably 0.45 to 0.55, even more preferably about 0.51;
R is 0.02 to 0.04, in particular 0.025 to 0.038, preferably 0.028 to 0.034, more preferably about 0.031;
X is 0.15 to 0.55, in particular 0.25 to 0.45, preferably 0.30 to 0.40, more preferably about 0.34;
Y is 0.05 to 1, in particular 0.05 to 0.50, preferably 0.09 to 0.40, preferably 0.09 to 0.30, more preferably 0.09 to 0.20, even more preferably about 0.14; and
Me is a monovalent metal ion such as a sodium ion or potassium ion, and is preferably a sodium ion.

95. The ferric octasaccharide of any one of embodiments 67-94, wherein the ferric octasaccharide has the formula:
{FeOOH, (C₆H₁₀O₆)_{T}—(C₆H₁₀O₅)_{Z}—(C₆H₁₃O₅)_{T}, (C₆H₈O₇)_{R}}, (H₂O)_{X}, (NaCl)_{Y}, wherein
T is 0.08 to 0.09, preferably about 0.085;
Z is 0.45 to 0.55, preferably about 0.51;
R is 0.028 to 0.034, preferably about 0.031;
X is 0.30 to 0.40, preferably about 0.34; and
Y is 0.05 to 1, in particular 0.05 to 0.50, preferably 0.09 to 0.40, preferably 0.09 to 0.30, more preferably 0.09 to 0.20, even more preferably about 0.14.

96. Ferric octasaccharide comprising iron complexed with an octasaccharide, wherein the ferric octasaccharide has the formula:
{FeOOH, (Octasaccharide)_{Q}}, that optionally contains a stabilizer and/or a metal chloride and/or H₂O, wherein
Q is 0.06 to 0.11, in particular 0.07 to 0.10, preferably 0.08 to 0.09, more preferably about 0.085.

97. The ferric octasaccharide of embodiment 96, wherein Q is 0.07 to 0.10, preferably 0.08 to 0.09, more preferably about 0.085.

98. The ferric octasaccharide of embodiment 96 or 97, wherein the ferric octasaccharide contains citric acid.

99. The ferric octasaccharide of any one of embodiments 96-98, wherein the ferric octasaccharide has the formula:
{FeOOH, (Octasaccharide)_{Q}, (C₆H₈O₇)_{R}}, that optionally contains a metal chloride and/or H₂O, wherein
Q is 0.06 to 0.11, in particular 0.07 to 0.10, preferably 0.08 to 0.09, more preferably about 0.085; and
R is 0.02 to 0.04, in particular 0.025 to 0.038, preferably 0.028 to 0.034, more preferably about 0.031.

100. The ferric octasaccharide of embodiment 99, wherein R is 0.025 to 0.038, preferably 0.028 to 0.034, more preferably about 0.031.

101. The ferric octasaccharide of any one of embodiments 96-100, wherein the ferric octasaccharide contains sodium chloride.

102. The ferric octasaccharide of any one of embodiments 96-101, wherein the ferric octasaccharide contains water.

103. The ferric octasaccharide of any one of embodiments 96-102, wherein the ferric octasaccharide has the formula:
{FeOOH, (Octasaccharide)_{Q}, (C₆H₈O₇)_{R}}, (H₂O)_{X}, (MeCl)_{Y}, wherein
Q is 0.06 to 0.11, in particular 0.07 to 0.10, preferably 0.08 to 0.09, more preferably about 0.085;
R is 0.02 to 0.04, in particular 0.025 to 0.038, preferably 0.028 to 0.034, more preferably about 0.031;
X is 0.15 to 0.55, in particular 0.25 to 0.45, preferably 0.30 to 0.40, more preferably about 0.34;
Y is 0.05 to 1, in particular 0.05 to 0.50, preferably 0.09 to 0.40, preferably 0.09 to 0.30, more preferably 0.09 to 0.20, even more preferably about 0.14; and
Me is a monovalent metal ion.

104. The ferric octasaccharide of embodiment 103, wherein X is 0.25 to 0.45, preferably 0.30 to 0.40, more preferably about 0.34.

105. The ferric octasaccharide of embodiment 103 or 104, wherein Y is 0.05 to 0.50, preferably 0.09 to 0.40, preferably 0.09 to 0.30, more preferably 0.09 to 0.20, even more preferably about 0.14.

106. The ferric octasaccharide of any one of embodiments 103-105, wherein the monovalent metal ion is a sodium ion or potassium ion.

107. The ferric octasaccharide of embodiment 106, wherein the monovalent metal ion is a sodium ion.

108. The ferric octasaccharide of any one of embodiments 103-107, wherein:
Q is 0.08 to 0.09, preferably about 0.085;
R is 0.028 to 0.034, preferably about 0.031;
X is 0.30 to 0.40, preferably about 0.34;
Y is 0.05 to 1, in particular 0.05 to 0.50, preferably 0.09 to 0.40, preferably 0.09 to 0.30, more preferably 0.09 to 0.20, even more preferably about 0.14; and
Me is a sodium ion.

109. The ferric octasaccharide of any one of embodiments 96-108, wherein the ferric octasaccharide has the formula:
{FeOOH, (C₆H₁₀O₆)_{T}—(C₆H₁₀O₅)_{Z}—(C₆H₁₃O₅)_{T}, (C₆H₈O₇)_{R}}, (H₂O)_{X}, (MeCl)_{Y}, wherein
T is 0.06 to 0.11, in particular 0.07 to 0.10, preferably 0.08 to 0.09, more preferably about 0.085;
Z is 0.25 to 0.75, in particular 0.35 to 0.65, preferably 0.45 to 0.55, even more preferably about 0.51;
R is 0.02 to 0.04, in particular 0.025 to 0.038, preferably 0.028 to 0.034, more preferably about 0.031;
X is 0.15 to 0.55, in particular 0.25 to 0.45, preferably 0.30 to 0.40, more preferably about 0.34;
Y is 0.05 to 1, in particular 0.05 to 0.50, preferably 0.09 to 0.40, preferably 0.09 to 0.30, more preferably 0.09 to 0.20, even more preferably about 0.14; and
Me is a monovalent metal ion such as a sodium ion or potassium ion, and is preferably a sodium ion.

110. The ferric octasaccharide of any one of embodiment 109, wherein:
T is 0.08 to 0.09, preferably about 0.085;
Z is 0.45 to 0.55, preferably about 0.51;
R is 0.028 to 0.034, preferably about 0.031;
X is 0.30 to 0.40, preferably about 0.34;
Y is 0.05 to 1, in particular 0.05 to 0.50, preferably 0.09 to 0.40, preferably 0.09 to 0.30, more preferably 0.09 to 0.20, even more preferably about 0.14; and
Me is a sodium ion.

111. The ferric octasaccharide of any one of embodiments 96-110, wherein the ferric octasaccharide has a weight average molecular weight in the range of 1,150 to 1,350 Da, preferably from 1,200 to 1,300 Da, more preferably from 1,225 to 1275 Da; e.g., of about 1250 Da.

112. The ferric octasaccharide of any one of embodiments 96-111, wherein the ferric octasaccharide has an “apparent” peak molecular weight (Mp measured by Gel Permeation Chromatography) in the range of 125,000 to 185,000 Da, preferably in the range 135,000 to 175,000 Da, more preferably 140,000 to 155,000 Da, e.g. in the range of 145,000 to 155,000 Da.

113. The ferric octasaccharide of any one of embodiments 96-112, wherein the ferric octasaccharide has a monosaccharide and disaccharide content of less than 10.0% by weight of the octasaccharide, preferably less than 3.0%, more preferably less than 1.0%; e.g., 0.1 to 0.5%, by weight of the octasaccharide.

114. The ferric octasaccharide of any one of embodiments 96-113, wherein the fraction of ferric octasaccharide with more than 9 monosaccharide units is less than 40% by weight of the octasaccharide, preferably less than 35%, more preferably less than 30%; e.g., 20 to 30%, by weight of the octasaccharide.

115. The ferric octasaccharide of any one of embodiments 96-114, wherein the proportion of molecules having 6-10 monosaccharide units is at least 40% by weight of the oligoisomaltoside molecules, preferably at least 45%; e.g., from 40 to 60% or from 45 to 55%, by weight of the octasaccharide.

116. The ferric octasaccharide of any one of embodiments 96-115, wherein the proportion of molecules having 6-10 monosaccharide units is higher by weight than the proportion of molecules having 3-6 monosaccharide units.

117. The ferric octasaccharide of any one of embodiments 96-116, wherein the dispersity (Mw/Mn) of the ferric octasaccharide is in the range of 1.05 to 1.4, preferably in the range of 1.1 to 1.3; e.g., at about 1.2.

118. The ferric octasaccharide of any one of embodiments 96-117, wherein the amount of reducing sugar in the ferric octasaccharide is 2.5% or less by weight of the octasaccharide, preferably 2.5% or less; preferably 1.0% or less; more preferably 0.5% or less; e.g. about 0.3%, by weight of the octasaccharide.

119. The ferric octasaccharide of any one of embodiments 96-118, wherein the amount of reducing sugar in the ferric octasaccharide prior to hydrogenation is (i) at least 10% or at least 15% and (ii) less than 35%; preferably no more than 30%; e.g. 10% to 30% or preferably 15 to 25%, by weight of the octasaccharide.

120. The ferric octasaccharide of any one of embodiments 96-119, wherein the ferric octasaccharide contains 10 to 50%; preferably 15 to 35; most preferably 20 to 30%; e.g., 20 to 25%, of iron by weight of the ferric octasaccharide.

121. The ferric octasaccharide of any one of embodiments 96-120, wherein the weight ratio of elemental iron to octasaccharide in the ferric octasaccharide is 10:90 to 50:50; preferably 15:85 to 45:55; most preferably 20:80 to 40:60; e.g., about 70:30.

122. The ferric octasaccharide of any one of embodiments 96-121, wherein the total amount of free iron in the ferric octasaccharide is 0.01% w/v or less; preferably less than 0.003% w/v, for a 100 mg/mL solution. 123. A pharmaceutical composition comprising the ferric octasaccharide of any one of embodiments 67-122 and a pharmaceutically acceptable carrier.

124. The pharmaceutical composition of embodiment 123, which is a solid, preferably powder, for reconstitution.

125. The pharmaceutical composition of embodiment 123, which is a ready-to use fluid or a fluid for dilution prior to use.

126. The pharmaceutical composition of any one of embodiments 123-125, which is suitable for subcutaneous administration.

127. The pharmaceutical composition of any one of embodiments 123-126, which comprises 1 to 25%; preferably 2 to 15%; most preferably 2.5 to 7.5 or 7.5 to 12.5; e.g., about 5% or about 10%, (w/v) of elemental iron.

128. The pharmaceutical composition of any one of embodiments 123-127, wherein the concentration of the iron complex compound is 25 to 300 mg/mL; preferably 50 to 200 mg/mL; most preferably 75 to 150 mg/mL; e.g. about 100 mg/mL of elemental iron.

129. The pharmaceutical composition of any one of embodiments 123-128, wherein the pH is 5.8 to 7.0; preferably 5.9 to 6.8; most preferably 5.9 to 6.6; e.g., 6.0 to 6.4.

130. The pharmaceutical composition of any one of embodiments 123-129, wherein the turbidity is below 2.0 NTU; preferably below 1.5 NTU; most preferably below 1.0 NTU; e.g. below 0.5.

131. The pharmaceutical composition of any one of embodiments 123-130, which has a viscosity of no greater than 60 cP.

132. The pharmaceutical composition of any one of embodiments 123-131, which has a shelf-life of at least 3 years at 25° C.

133. The ferric octasaccharide of any one of embodiments 67-122 or the pharmaceutical composition of any one of embodiments 123 to 132 for use in a method of therapy of iron deficiency in a human or non-human subject.

134. The ferric octasaccharide or the pharmaceutical composition for use of embodiment 133, wherein the non-human subject is a companion animal.

135. The ferric octasaccharide or the pharmaceutical composition for use of embodiment 134, wherein the companion animal is a canine, feline or equine.

136. The ferric octasaccharide or the pharmaceutical composition for use of embodiment 134, wherein the companion animal is a dog or a cat.

137. The ferric octasaccharide or the pharmaceutical composition for use of embodiment 134, wherein the companion animal is a dog.

138. The ferric octasaccharide or the pharmaceutical composition for use of any one of embodiments 134-137, wherein the companion animal has a reticulocyte haemoglobin content (CHr)/reticulocyte haemoglobin equivalent (RET-He) of 20 pg or less.

139. The ferric octasaccharide or the pharmaceutical composition for use of any one of embodiments 134-138, wherein the iron deficiency is iron deficiency anaemia.

140. The ferric octasaccharide or the pharmaceutical composition for use of any one of embodiments 134-139, wherein the companion animal, preferably a dog or a cat, has a haematocrit (HCT/PCV) of less than 35%.

141. The ferric octasaccharide or the pharmaceutical composition for use of any one of embodiments 134-140, wherein the companion animal, preferably a dog or a cat, has a haemoglobin concentration (Hb) of less than 12 g/dL.

142. The ferric octasaccharide or the pharmaceutical composition for use of any one of embodiments 134-141, wherein the companion animal, preferably a dog or a cat, has a mean corpuscular volume (MCV) of less than 60 fL.

143. The ferric octasaccharide or the pharmaceutical composition for use of any one of embodiments 134-142, wherein the companion animal, preferably a dog or a cat, has a mean corpuscular haemoglobin concentration (MCHC) of 30 g/dL or less.

144. The ferric octasaccharide or the pharmaceutical composition for use of any one of embodiments 134-143, wherein the dose is 5 to 100 mg per kg body weight; preferably 10 to 60 mg per kg body weight; most preferably 15 to 25 mg per kg body weight; e.g., about 20 mg per kg body weight.

145. The ferric octasaccharide or the pharmaceutical composition for use of any one of embodiments 134-144, wherein the dose is up to 50 mg iron/kg body weight; preferably up to 30 mg iron/kg body weight; most preferably up to 20 mg iron/kg body weight.

146. The ferric octasaccharide or the pharmaceutical composition for use of any one of embodiments 134-145, wherein a single dose is administered.

147. The ferric octasaccharide or the pharmaceutical composition for use of any one of embodiments 134-146, wherein the dose is provided as a single (1) administration, preferably injection and especially subcutaneous injection.

148. The ferric octasaccharide or the pharmaceutical composition for use of any one of embodiments 134-146, wherein the dose is provided as 2, 3, or more administrations, preferably injections and especially subcutaneous injections.

149. The ferric octasaccharide or the pharmaceutical composition for use of any one of embodiments 134-145, wherein more than one dose is administered.

150. The ferric octasaccharide or the pharmaceutical composition for use of embodiment 149, wherein the more than one dose is a dose of up to 50 mg iron/kg body weight; preferably up to 30 mg iron/kg body weight; most preferably up to 20 mg iron/kg body weight.

151. The ferric octasaccharide or the pharmaceutical composition for use of embodiment 149 or 150, wherein two consecutive doses are administered within 1 month; preferably within 2 weeks; most preferably within 1 week.

152. The ferric octasaccharide or the pharmaceutical composition for use of any one of embodiments 134-151, wherein the administration is subcutaneous administration.

153. The ferric octasaccharide or the pharmaceutical composition for use of embodiment 152, wherein the site of subcutaneous administration is in the area laterally above the dorsal plane behind the shoulder blades over the ribs or in the dorsal paralumbar region.

154. The ferric octasaccharide or the pharmaceutical composition for use of embodiment 134, wherein a single dose of 20 mg/kg body weight is administered subcutaneously to a dog or a cat.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from one particular value, and/or to another particular value. When such a range is expressed, another embodiment includes from the one particular value and/or to the other particular value. It will be appreciated by one of ordinary skilled in the art that such values are as accurate as the method used to measure it and thus the values disclosed herein will be understood to be associated with error margins.

Similarly, when values are expressed as approximations, by the use of the antecedent “about,” it will be understood that the particular value forms another embodiment. As used herein, the term “about”, refers to the indicated value of the variable and to all values of the variable that are within the experimental error of the indicated value (e.g., within the 95% confidence interval for the mean) or within 10 percent of the indicated value, whichever is greater.

### EXAMPLES

### Example 1—Production of Ferric Oligoisomaltoside

Ferric oligoisomaltosides were manufactured from different dextran fractions that were combined and fractionated by ultrafiltration. The dextran fractions were produced from intermediate dextran. In several stages, the starting material was hydrolysed to a lower molecular weight, fractionated by ultrafiltration and filtrated until the desired molecular weight distribution was achieved. The oligoisomaltoses were finally hydrogenated and ion exchanged and then reacted with ferric chloride for complex formation.

### Manufacturing of Ferric Octasaccharide

### Carbohydrate Fractionation

A pre-hydrolysed dextran fraction with a weight average molecular weight estimated to be around 2 kDa to 5 kDa (4872 kg) was hydrolysed by addition of conc. HCl to pH 1.5 and stirring at 90° C. until the chromatographic peak ascent conformed to that of an external dextran standard (M_{w} less than 2 kDa). The solution was cooled to 28° C. and neutralised with NaOH. The solution was purified by diafiltration against water at 51° C. until a narrow distribution with a molecular weight of 1150 to 1350 Da and a polydispersity of about 1.2 was achieved. The amount of reducing sugar determined by Somogyi's reagent was 21%.

### Carbohydrate Hydrogenation

The resulting fraction (1652 kg) was treated with sodium borohydride at pH 10.2 at 28° C. The level of reducing sugar measured by Somogyi reagent was less 0.02%. The solution was acidified to pH 2.0 with conc. HCl and stirred for 3 hrs and then pH was adjusted to 4.6 with NaOH.

The solution was de-ionized by ion exchange to give a product solution containing octasaccharide with a conductivity of below 500 μS/cm.

The weight average molecular weight (Mw) of the octasaccharide was determined to be 1,235 Da.

### Iron Complexation

560 kg of octasaccharide and 240 kg of elementary iron from ferric chloride were used for complex formation. A liquid solution containing 70 kg of octasaccharide was added to the complexation reactor. Water for Injection (WFI) was added while stirring followed by FeCl₃, 6 H₂O eq. to 240 kg of elementary iron. 600 kg of Na₂CO₃ (aq.) was added while stirring after NaOH was added to reach a pH of about 10.5. The solution was heated to above 100° C. until it turned into a black or dark brown colloidal solution. Subsequently the solution was neutralized using HCl and filtered. The solution was purified by membrane filtration to remove residues of unbound octasaccharide, free iron, and inorganic salts. Citric acid monohydrate dissolved in sodium hydroxide was added. The pH was adjusted to 5.6 and the resulting solution was spray-dried resulting in a black to dark brown powder.

The “apparent” peak molecular weight (Mp) of the ferric octasaccharide complex was determined to be 147,121 Da, with Mw/Mn (dispersity) being calculated as 1.15. To this end, the composition was diluted to 0.1% iron (1 mg/mL) in eluent and the chromatogram was measured on GPC (plus reference standards, dextrans and iron dextrans). Mp was read of the chromatogram, and Mn and Mw were calculated using the calibration curve.

### Complex Strength

Absorbance (287.3 nm) after acid hydrolysis is proportional to amount of complex-bound iron. Absorbance is measured overtime. T½ is the duration of time until half of the original complex-bound iron has been released. The sample was diluted to 0.02%=200 mg/L of iron. 5 mL diluted sample was hydrolysed in 100 mL 0.25 M HCl with 0.9 g NaCl.

The complex strength of the resulting ferric octasaccharide was found to be high. When subjected to hydrochloric acid hydrolysis under test conditions, it took 40 hours for half of the complex to dissociate into its component parts; iron and carbohydrate. This T½ is lower than the T½ that is usually observed for iron dextran and iron dextran glucoheptonic acid complexes (which is typically in the range of 70 to 80 hours, depending on the type of dextran and complex). At the same time, the T½ I is higher than the T½ that is observed for complexes with weakly bound iron such as in iron sucrose or iron gluconate.

### Free Iron

The amount of free iron in the composition comprising the iron carbohydrate complex (i.e., colloidal iron of less than 12-14 kDa in size) was determined by dialyzing the iron to clean water and measuring the amount of iron in the dialysate by atom absorption spectroscopy.

3 mL native composition was dialysed in dialysis tube in 20 mL water for 24 hours. Then the amount of iron in the dialysate was determined. % free iron is calculated as iron in dialysate relative to the total amount of iron in 3 mL of composition.

The amount of free iron for ferric octasaccharide was determined to be below 0.003% w/v, which indicates that the product is safe.

### Iron and Carbohydrate Content

For determining the carbohydrate content, the composition comprising the iron carbohydrate complex was diluted and all glucose in the complex was freed and bound with anthron-HCl. The amount of glucose was measured by spectrophotometry.

The carbohydrate content in a composition of ferric octasaccharide with an iron concentration of 10% w/v was found to be as high as 22% w/v. This is significantly higher than the carbohydrate content that is usually observed for iron dextran and iron dextran glucoheptonic acid complexes (which can be as low as half of the value for ferric octasaccharide, depending on the type of dextran and complex). Put another way, the ratio of iron to carbohydrate in ferric octasaccharide is lower than in typical iron dextran and iron dextran glucoheptonic acid complexes. This in turn means that individual iron particles (akageneite particles) are better protected by glucose units, leading to better physical stability.

### Manufacturing of Further Iron Oligoisomaltosides

Further iron oligoisomaltosides were manufactured using essentially the same process steps but with the objective of producing iron complexes with oligoisomaltosides that have a different molecular weight distribution than the ferric octasaccharide. For instance, in an alternative process, the fractionation was conducted such that the resulting oligoisomaltosides had a lower weight average molecular weight in the range of 850 to 1,150 Da (measured by GPC). For instance, one such oligoisomaltoside had a weight average molecular weight of 1,047 Da. The corresponding iron complexes were found to have a complex strength (T½=33 to 37 hours) comparable to that of ferric octasaccharide, but the carbohydrate content (about 18% w/v) was slightly but significantly lower than in ferric octasaccharide when measured at the same iron concentration.

### Example 2—Study to Evaluate the Tolerance, Safety, Pharmacokinetics (PK) and Pharmacodynamics (PD) of Ferric Oligoisomaltoside Injected Subcutaneously (SC) and Intramuscularly (IM) in Healthy Laboratory Dogs (Without ID or IDA)

### Study Objectives

This pilot study was designed to explore the treatment of dogs with ferric oligoisomaltoside. Specific objectives included determining iron pharmacokinetic profiles in serum and urine; determining the pharmacodynamics profile of haemoglobin, reticulocyte count, calcium, ferritin, unsaturated iron binding capacity, total iron binding capacity, and transferrin saturation; evaluating the injection site reactions through routine monitoring; and determining the tolerance of dogs to injection with the compound and the compounds' early safety profile based on clinical pathology.

This single-site, non-clinical laboratory study in dogs involved four dose groups in an unblinded, randomised parallel design. Fourteen (14) males and 14 females were acclimated to study conditions for seven days during which they were subjected to body weight measurement, physical examination, blood collection for haematology and clinical chemistry analysis, urine collection for urinalysis and twice daily clinical observations.

Following acclimation, 24 Beagle dogs (12 males and 12 females; weighing between 7.2 to 12.4 kg) were randomised into one of four sex-balanced dose groups of six dogs each. Dogs were dosed once as per the following table with ferric oligoisomaltoside (a compound produced according to example 1; 100 mg/mL elemental iron; pH=6.3). The intended 1× dose is 20 mg/kg, equal to 0.2 mL/kg. In the T3 group, the dose is 100 mg/kg, equal to 1 mi/kg. For instance, a dog with a body weight of 10.3 kg in group T2 was dosed with 6.2 mL of ferric oligoisomaltoside.

| Group | n | Route | Dose |
|---|---|---|---|
| T1 (1X) | 3M, 3F | SC | 20 mg/kg |
| T2 (3X) | 3M, 3F | SC | 60 mg/kg |
| T3 (5X) | 3M, 3F | SC | 100 mg/kg |
| T4 (1X) | 3M, 3F | IM | 20 mg/kg |

To define the location of injection site evaluations, a thin outline of the left dorsal paralumbar region, encompassing both IM and SC sites, was shaved prior to Day 0. This outline ensured all technicians performed injection site evaluations within a consistent area. Shaving of the injection site was not permitted. Ferric oligoisomaltoside was administered by SC (left dorsal paralumbar region) or IM (left dorsal paralumbar epaxial muscles) injection.

For SC injections (groups T1, T2, and T3):
The dose was drawn into the syringe and any air evacuated;
The dog was restrained to prevent movement during injection;
The skin over the left dorsal paralumbar region was tented;
The needle was inserted into the SC space and negative pressure applied to the plunger to confirm the needle was in the SC space and not a vascular area;
The entire intended dose was injected and the needle removed from the skin;
Pain was evaluated immediately after needle placement while test article was being injected;
The dog was returned to its pen, and the handler changed gloves between each dog.

For IM injections (group T4):
The dose was drawn into the syringe and any air evacuated;
The dog was restrained to prevent movement during injection;
The left dorsal paralumbar (epaxial) muscle was identified;
The needle was inserted into the muscle and negative pressure applied to the plunger to confirm the needle was within the muscle and not a vascular area;
The entire intended dose was injected, and the needle was removed from the muscle;
Pain was evaluated immediately after needle placement while test article was being injected;
The dog was returned to its pen, and the handler changed gloves between each dog.

Study variables were assessed as follows:
Tolerance to injection was assessed during dose administration;
Serial blood collections for pharmacokinetic and pharmacodynamic analyses were performed at 0¹, 0.5, 1, 2, 4, 8, 24, 48, 72, 120, 168, 240, 336, and 504 h post-dosing; ¹ Pre-dose (0 h) PK and PD samples were collected on Day −1 for all dogs.
Urine for pharmacokinetic analysis was collected at intervals of 0 to 8, 8 to 24, 24 to 48, and 48 to 72 h post-dosing;
Clinical observations were performed twice daily (at least 6 h apart) from the first day of acclimation until the last day of the study;
Injection site evaluations were performed on Day 0 prior to dosing, at 1, 2, and 6 h (all ±15 min), at 24, 48, and 72 h (±1 h), and on Days 4, 7, 10, 14, and 21 (±1 h post-dosing);
Physical examinations were performed once during acclimation (Day −6) and on Day 21;
Body weights were measured once during acclimation (Day −7), on Day 0 (prior to dosing), and on Days 7 and 21;
Blood for clinical pathology was collected once during acclimation (Day −5), and on Days 2, 7, and 21;
Urine was collected for urinalysis once during acclimation (Day −7 or Day −5), on Days 1 or 2 and Days 7 or 8 and Days 20 or 21;
Food consumption was measured from the first day of acclimation until the end of the study.

### Results

Significant dose group effects suggesting a pharmacokinetic dose relationship were identified after baseline adjusting serum iron concentrations to determine AUCₜₗₐₛₜ and Cₘₐₓ. Baseline corrected AUCₜₗₐₛₜ values were significantly lower in groups T1 and T4 as compared to groups T2 and T3. Baseline corrected Cₘₐₓ values were significantly lower in group T1 as compared to group T3; no other comparisons were statistically significant.

The main findings of the study of are summarised as follows:
SC (T1) injection gave comparatively less pain than IM (T4 at a 20 mg/kg dose). SC injection of 20 mg/kg (T1) gave comparatively less pain than SC injection of 100 mg/kg (T3).

| Pain | Number of dogs by dose group | | | | |
|---|---|---|---|---|---|
| Score | T1 | T2 | T3 | T4 | Subtotal |
| 0 | 3 | 3 | 1 | 1 | 8 |
| 1 | 0 | 0 | 1 | 1 | 2 |
| 2 | 2 | 2 | 0 | 1 | 5 |
| 3 | 1 | 1 | 4 | 3 | 9 |

*0: no pain: dog unaffected;
1: mild pain: skin flinch;
2: moderate pain; dog turns to the injection site;
3: severe pain: dog vocalizes and/or attempts to evade restraint and/or becomes aggressive

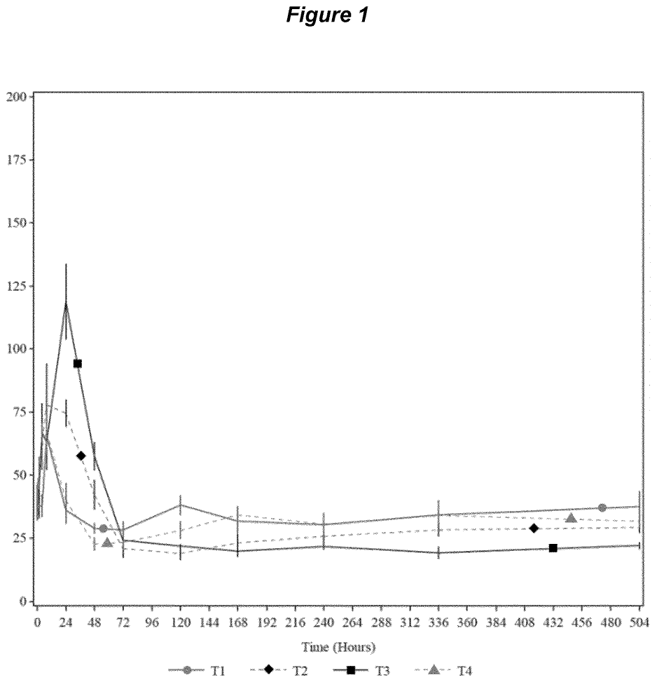

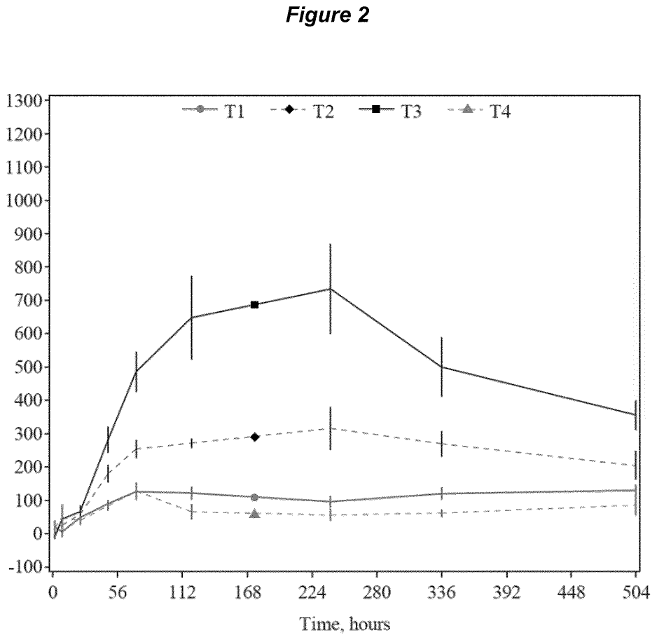

There were no documented post-dosing occurrences of heat, pain, or swelling at the injection sites for any dog throughout the study and no other drug-related adverse event were reported.
But surprisingly, 20 mg/kg SC injection T1 had essentially the same pharmacokinetic profile of serum iron as 20 mg/kg IM injection T4, see FIG. 1.
And surprisingly, a dose-related increase in ferritin was observed, with T1 and T4 profiles basically giving the same ferritin response, and with T2 and T3 giving a proportionally larger response; see FIG. 2.

### Conclusions

Overall, ferric oligoisomaltoside was well tolerated at all dosage levels and by both administration routes. Dogs remained in good health over the course of the study.

PK profiles defined AUCₜₗₐₛₜ, and Cₘₐₓ for both serum and urine iron concentrations after injection of ferric oligoisomaltoside in dogs. In serum, statistically significant dose effects on serum iron were detected using baseline adjusted AUCₜₗₐₛₜ and Cₘₐₓ parameters. AUCₜₗₐₛₜ and Cₘₐₓ were not significantly different in the T4 (1×IM) and T1 (1×SC) groups, indicating that the route of administration of ferric oligoisomaltoside did not significantly impact PK profile. The route of administration (IM vs SC) did not influence any of the PD parameters either, as there were no significant differences observed between the T4 (1×IM) and T1 (1×SC) groups. Comparable PD profiles were observed in the T4 (1×IM) and T1 (1×SC) groups.

In urine, cumulative iron excretion demonstrated a short-term dose effect, which dissipated by the end of the sampling period. The route of administration did not have a meaningful impact on cumulative iron excretion in urine.

Reticulocyte counts and calcium, and most PD parameters demonstrated consistent, short-term, dose proportional effects. Ferritin and TSAT increased in a dose-proportional manner. Even the T1 (1×SC) and T4 (1×IM) treatment groups demonstrated effects on ferritin and TSAT.

Because ferritin was found to increase in a dose-dependent manner and ferritin is expected to be increased when an iron-deficient subject is treated with a parenteral iron compound, it is reasonable to expect that subcutaneous injection of ferric oligoisomaltoside will be suitable to treat iron deficiency and iron deficiency anaemia in dogs and other companion animals.

### Example 3—Absorption of Ferric Octasaccharide after Subcutaneous (SC) or Intramuscular (IM) Injection in Rabbits

### Study Objectives

0.4 mL/kg bodyweight of a 10% (w/v) composition comprising ferric octasaccharide was injected IM or SC into a rabbit leg, the other leg being used as control. The rabbit was euthanised either after 24 hours or after 7 days.

In the case of IM injection, absorption was evaluated by checking how much iron remains in the injected muscle—by visual and quantitative evaluation. For quantitative evaluation, the muscle was homogenised, and subjected to destruction by NaOH followed by H2SO4/HNO3 with boiling at 140° C. for 20 hours. Then, the amount of iron in the destructed sample was determined by Atomic Absorption Spectrometry (AAS; see the British Pharmacopoeia, current edition: Iron dextran injection, “Test for Iron Absorption”—modified), and the fraction of iron remaining at the site of injection—i.e., the iron not absorbed from the site of injection—was calculated. Thereby the fraction of iron absorbed was calculated as 100% minus the fraction of iron remaining at the site of injection.

In the case of SC injection, the same procedure as for IM injection was followed with one exception: the entire muscle and the skin were analysed, i.e., the muscle was not skinned prior to analysis.

### Results

Ferric octasaccharide was well and quickly absorbed from the injection site, both when administered intramuscularly or subcutaneously. After 7 days, 98-99% of the dose was absorbed from the injection site and there was no significant difference observed between subcutaneous and intramuscular injection. Within the shorter time frame of 24 hours after injection, the absorption of iron given intramuscularly was found to be essentially complete (99.4%) while the iron given subcutaneously had not yet been absorbed in full (96.3%).

### Conclusions

7 days after injection, the iron in ferric octasaccharide was found to be completely absorbed from the injection site. There was no significant difference observed between subcutaneous and intramuscular injection. Surprisingly, the absorption of iron within the first 24 hours from subcutaneously administered ferric octasaccharide was observed to be almost as quick as when administered intramuscularly (96.3% vs. 99.4%). While intramuscular administration is expected to lead to a relatively quick absorption of iron, the absorption of iron from a subcutaneously administered iron complex compound is usually expected to be significantly slower.

### REFERENCES

A number of publications are cited above in order to more fully describe and disclose the invention and the state of the art to which the invention pertains. Full citations for these references are provided below. The entirety of each of these references is incorporated herein.
Bohn, A. A. (2013). Diagnosis of Disorders of Iron Metabolism in Dogs and Cats. Veterinary Clinics: Small Animal Practice, 43(6), 1319-30.
Cohen-Solal, A., Leclercq, C., Deray, G., Lasocki, S., Zambrowski, J. J., Mebazaa, A., Groote, P., Damy, T., & Galinier, M. (2014). Iron deficiency: an emerging therapeutic target in heart failure. Heart, 100(18), 1414-20.
Cook, A. K. & Kvitko-White, H. L. (2014). DVM360—Case report Jun. 1, 2014: A 5-year-old golden retriever with IDA, http://veterinarymedicine.dvm360.com/case-report-5-year-old-golden-retriever-with-ida (accessed Sep. 3, 2019).
Dignass, A. U., Gasche, C., Bettenworth, D., Birgegård, G., Danese, S., Gisbert, J. P., . . . & Vavricka, S. (2015). European consensus on the diagnosis and management of iron deficiency and anaemia in inflammatory bowel diseases. Journal of Crohn's and Colitis, 9(3), 211-22.
Fry, M. M. & Kirk, C. A. (2006). Reticulocyte indices in a canine model of nutritional iron deficiency. Veterinary Clinical Pathology, 35(2), 172-81.
Fuchs, J., Moritz, A., Grußendorf, E., Lechner, J., Neuerer, F., Nickel, R., Rieker, T., Schwedes, C., DeNicola, D. B., Russell, J., Bauer, N. (2017). Evaluation of reticulocyte hemoglobin content (RET-He) in the diagnosis of iron deficient erythropoiesis in dogs. Veterinary Clinical Pathology, 46(4), 558-68.
Giger, U. (2005). In Ettinger, S. J. & Feldman, E. C.—Textbook of Veterinary Internal Medicine. Chapter 270: Regenerative anemias caused by blood loss or hemolysis, 1888-90. 6ₜₕ ed. Elsevier.
Giger, U. (2010). In Weiss, D. J. & Wardrop, K, J.—Schalm's Veterinary Hematology. Chapter 28: Hereditary Erythrocyte Enzyme Abnormalities, 183-5. 6ₜₕ ed. Wiley-Blackwell.
Harvey, J. W., French, T. W., & Meyer, D. J. (1982). Chronic iron deficiency anemia in dogs. Journal of the American Animal Hospital Association, 18(6), 948-60.
Lund, E. (2007). Population analyses of anemia in Pets—Banfield DataSavant analysis, 2007.
https://www.banfield.com/getmedia/8b7bab32-5503-43f1-aebd-4fdf558e5d0d/3_5-Population-Analysis-of-Anemia-in-Pets (accessed Sep. 3, 2019).
Merck Veterinary Manual, Reference Guides, Hematologic Reference Ranges. https://www.merckvetmanual.com/special-subjects/reference-guides/hematologic-reference-ranges (accessed Aug. 29, 2019).
Naigamwalla, D. Z., Webb, J. A., & Giger, U. (2012). Iron deficiency anemia. The Canadian Veterinary Journal, 53(3), 250-6.
Nelson, R. W. & Couto, C. G. (1998). Small Animal Internal Medicine. Chapter 85, 1171-2. 2_{nd} Ed. Elsevier.
Olver, C. S., Andrews, G. A., Smith, J. E., and Kaneko, J. J. (2010). In Weiss, D. J. & Wardrop, K, J.—Schalm's Veterinary Hematology. Chapter 20: Erythrocyte Structure and Function, 123-9. 6ₜₕ ed. Wiley-Blackwell.
Plumb, D. C. (2008). Plumb's Veterinary Drug Handbook. Pages 382-3 and 493-4. 6th ed. Blackwell Publishing.
Preusser, L. C., Fryer F. M., Gerhardt, A., Yanhui Hu, Delgado-Herrera L., Melnick, J. Z., Williams, L. A., Cox, B. F., and Reinhart, G. A. (2005). Clinical and Experimental Pharmacology and Physiology, 32, 1020-1026
Prins, M., van Leeuwen, M. W., Teske, E. (2009). Stability and reproducibility of ADVIA 120-measured red blood cell and platelet parameters in dogs, cats, and horses, and the use of reticulocyte haemoglobin content (CHr) in the diagnosis of iron deficiency. Tijdschrift voor Diergeneeskunde (Journal of Veterinary Medicine), 134(7), 272-8.
Schaefer, D. M. W. & Stokol, T. (2015). The utility of reticulocyte indices in distinguishing iron deficiency anemia from anemia of inflammatory disease, portosystemic shunting, and breed-associated microcytosis in dogs. Veterinary Clinical Pathology, 44(1), 109-19.
Steinberg, J. D. & Olver, C. S. (2005). Hematologic and biochemical abnormalities indicating iron deficiency are associated with decreased reticulocyte hemoglobin content (CHr) and reticulocyte volume (rMCV) in dogs—Veterinary Clinical Pathology, 34(1), 23-7.
Thrall, M. A. & Gillespie, B. 9 (2011). Diagnosis of Iron Deficiency Anemia, VetCom Winter 2011
Tvedten, H. (2010). In Weiss, D. J. & Wardrop, K, J.—Schalm's Veterinary Hematology. Chapter 24: Laboratory and Clinical Diagnosis of Anemia, 152-9. 6ₜₕ ed. Wiley-Blackwell.
Weiser, G. (2015). In Tilley, L. P. & Smith Jr, F. W.—Blackwell's Five-Minute Veterinary Consult: Canine and feline. Anemia: Iron-Deficiency, page 83. 6ₜₕ ed. John Wiley & Sons.
Weiss, D. J. (2010). In Weiss, D. J. & Wardrop, K, J.—Schalm's Veterinary Hematology. Chapter 26: Iron and Copper Deficiencies and Disorders of Iron Metabolism, 167-71. 6ₜₕ ed. Wiley-Blackwell.

1. An iron complex compound for subcutaneous use in a method of therapy of iron deficiency in a dog.

2. The iron complex compound for the use of claim 1, wherein iron deficiency is iron deficiency anaemia.

3. The iron complex compound for the use of claim 1 or claim 2, wherein the method comprises administering a dose of 5 to 100 mg/kg body weight, preferably 10 to 60 mg/kg body weight, most preferably 15 to 25 mg/kg body weight of elemental iron.

4. The iron complex compound for the use of any one of claims 1 to 3, wherein the method comprises administering a dose of 20 mg/kg bodyweight of elemental iron.

5. The iron complex compound for the use of any one of claims 1 to 4, wherein the iron complex compound is an iron carbohydrate complex.

6. The iron complex compound for the use of any one of claims 1 to 5, wherein the iron complex compound is an iron oligoisomaltose complex or an iron oligoisomaltoside complex.

7. The iron complex compound for the use of any one of claims 1 to 6, wherein the site of subcutaneous administration is in the dorsal paralumbar region.

8. The iron complex compound for the use of any one of claims 1 to 7, wherein the dog has a haematocrit (HCT/PCV) of less than 35%.

9. The iron complex compound for the use of any one of claims 1 to 8, wherein the dog has a haemoglobin concentration (Hb) of less than 12 g/dL.

10. The iron complex compound for the use of any one of claims 1 to 9, wherein the dog has a mean corpuscular volume (MCV) of less than 60 fL.

11. The iron complex compound for the use of any one of claims 1 to 10, wherein the dog has a mean corpuscular haemoglobin concentration (MCHC) of 30 g/dL or less.

12. The iron complex compound for the use of any one of claims 1 to 11, wherein the dog has a reticulocyte haemoglobin content (CHr)/reticulocyte haemoglobin equivalent (RET-He) of 20 pg or less.

13. The iron complex compound for the use of any one of claims 1 to 12, wherein the method comprises administering a dose of the iron complex compound and the dose is provided as a single subcutaneous injection.

14. The iron complex compound for the use of any one of claims 1 to 13, wherein the method comprises administering a fluid pharmaceutical composition comprising the iron complex compound and a pharmaceutically acceptable carrier, wherein the concentration of the iron complex compound in the fluid pharmaceutical composition is 25 to 300 mg/mL, preferably 50 to 200 mg/mL, most preferably 75 to 150 mg/mL, e.g., about 100 mg/mL of elemental iron.

15. The iron complex compound for the use of claim 14, wherein the subcutaneous injection volume is 0.5 to 5 mL.
